(19) United States
(12) Patent Application Publication
(10) Pub. No.: US 2008/0312281 A1
(43) Pub. Date: Dec. 18, 2008

(54) G-Protein Coupled Receptor (Gpr116) Agonists and Use Thereof for Treating Obesity and Diabetes

(76) Inventors: Matthew Colin Thor Fyfe, Oxford (GB); Lisa Sarah Bertram, Oxford (GB); Gerard Hugh Thomas, Oxford (GB); Geoffrey Martyn Williams, Oxford (GB)

Correspondence Address: OSI Pharmaceuticals, 41 Pinelawn Rd., Melville, NY, 10017, US

(21) Appl. No.: 11/794,219

(22) PCT Filed: Dec. 23, 2005
(86) PCT No.: PCT/GB05/50264

§ 371 Date: Aug. 4, 2008

(30) Foreign Application Priority Data
Dec. 24, 2004 (GB) 0428212.5
Jun. 30, 2005 (GB) 0513255.0

(51) Int. Cl. A61K 31/44; C07D 401/00; A61P 3/10

(52) U.S. Cl. 514/318; 546/208

(57) ABSTRACT

Compounds of formula (I), or pharmaceutically acceptable salts thereof, are agonists of GPR116 and are useful for the treatment of obesity, and for the treatment of diabetes.

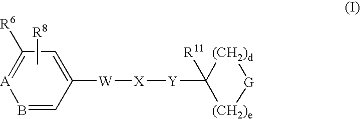

### BACKGROUND OF THE INVENTION

The present invention is directed to G-protein coupled receptor (GPCR) agonists. In particular, the present invention is directed to agonists of GPR116 that are useful for the treatment of obesity, e.g. as regulators of satiety, and for the treatment of diabetes.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycemic agents and insulin, but there is a continuing need for novel antidiabetic agents, particularly ones that may be better tolerated with fewer adverse effects.

Similarly, metabolic syndrome (syndrome X) which is characterized by hypertension and its associated pathologies including atherosclerosis, lipidemia, hyperlipidemia and hypercholesterolemia have been associated with decreased insulin sensitivity which can lead to abnormal blood sugar levels when challenged. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR116 is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR116 is expressed in the pancreas, small intestine, colon and adipose tissue. The expression profile of the human GPR116 receptor indicates its potential utility as a target for the treatment of obesity and diabetes.

International patent application WO2005/061489 (published after the priority date of the present application) discloses heterocyclic derivatives as GPR116 receptor agonists.

The present invention relates to agonists of GPR116 which are useful for the treatment of obesity, e.g. as regulators of satiety, and for the treatment of diabetes.

### SUMMARY OF THE INVENTION

Compounds of formula (I):

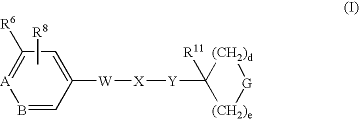

or pharmaceutically acceptable salts thereof, are agonists of GPR116 and are useful for the prophylactic or therapeutic treatment of obesity, and for the treatment of diabetes.

### DETAILED DESCRIPTION OF THE INVENTION

The present invention is directed to a compound of formula (I), or a pharmaceutically acceptable salt thereof:

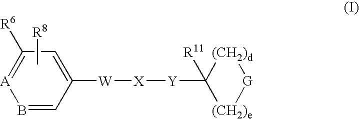

wherein one of A and B is nitrogen and the other is CR¹;

W and Y are independently a bond, an unbranched or a branched C₁₋₃ alkylene or an unbranched or a branched C₂₋₃ alkenylene;

X is selected from CH₂, O, S, CH(OH), CH(halogen), C(O), C(O)O, C(O)S, SC(O), C(O)CH₂S, C(O)CH₂C(OH), C(O)CH₂C(O), OC(O), NR⁵, CH(NR⁵R⁵⁵), C(O)NR², S(O) and S(O)₂;

G is CHR³, N—C(O)OR⁴, N—C(O)NR⁴R⁵, N—C₁₋₄alkylene-C(O)OR⁴, N—C(O)C(O)OR⁴, N—S(O)₂R⁴, N—C(O)R⁴ or N—P(O)(O-Ph)₂; or N-heterocyclyl or N-heteroaryl, either of which may optionally be substituted by one or two groups selected from C₁₋₄alkyl, C₁₋₄alkoxy or halogen;

R¹ is hydrogen, halogen, cyano, C(O)NH₂, C₁₋₄alkyl, SO₂C₁₋₄alkyl, SOC₁₋₄alkyl or SC₁₋₄alkyl;

R² is hydrogen or C₁₋₄ alkyl;

R³ is C₃₋₆ alkyl;

R⁴ is C₁₋₈ alkyl, C₂₋₈ alkenyl or C₂₋₈ alkynyl, any of which may be optionally substituted by one or more halo atoms, NR⁵R⁵⁵, OR⁵, C(O)OR⁵, OC(O)R⁵ or cyano, and may contain a CH₂ group that is replaced by O or S; or a C₃₋₇cycloalkyl, aryl, heterocyclyl, heteroaryl, C₁₋₄alkyleneC₃₋₇cycloalkyl, C₁₋₄alkylenearyl, C₁₋₄alkyleneheterocyclyl or C₁₋₄alkyleneheteroaryl, any of which may be substituted with one or more substituents selected from halo, C₁₋₄ alkyl, C₁₋₄ fluoroalkyl, OR⁵, CN, NR⁵R⁵⁵, SO₂Me, NO₂ or C(O)OR⁵;

R⁵ and R⁵⁵ are independently hydrogen or C₁₋₄alkyl; or taken together R⁵ and R⁵⁵ may form a 5 or 6 membered heterocyclic ring;

R⁶ is hydrogen, halogen, CN, C₁₋₄alkyl, C₁₋₄alkoxy, ethynyl, C(O)NR⁷R⁷⁷ or C₁₋₄alkyleneS(O)_{f};

R⁷ and R⁷⁷ are independently hydrogen or C₁₋₄alkyl; or taken together R⁷ and R⁷⁷ may form a 5 or 6 membered heterocyclic ring;

R⁸ is hydrogen, halogen, CN, C₁₋₄alkyl or C₁₋₄alkoxy;

R¹¹ is hydrogen or hydroxy;

d is 0, 1, 2 or 3;

e is 1, 2, 3, 4 or 5;

with the proviso that d+e is 2, 3, 4 or 5; and

f is 0, 1 or 2.

In one embodiment of the invention the compound of formula (I) is a compound of formula (Ia), or a pharmaceutically acceptable salt thereof:

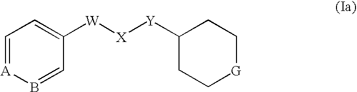

wherein one of A and B is nitrogen and the other is CR¹;

W and Y are independently a bond, C₁₋₃ alkylene or C₂₋₃ alkenylene;

X is selected from CH₂, O, S, CO, CO₂, COS, SCO, COCH₂S, COCH₂CO, OCO, CONR², SO and SO₂;

G is CHR³, NCOOR⁴, or NCONR⁴R⁵;

R¹ is hydrogen, halogen, cyano or C₁₋₄ alkyl;

R² is C₁₋₄alkyl;

R³ is C₃₋₆ alkyl;

R⁴ is C₁₋₆ alkyl, C₂₋₆ alkenyl or C₂₋₆ alkynyl optionally substituted by one or more fluoro atoms or cyano, C₃₋₇ cycloalkyl, or aryl optionally substituted with C₁₋₄alkyl, C₁₋₄alkoxy, halogen, CF₃, nitro, cyano, or CO₂C₁₋₄alkyl; and

R⁵ is hydrogen or C₁₋₄ alkyl.

The molecular weight of the compounds of formula (I) is preferably less than 800, more preferably less than 600, even more preferably less than 500.

A is preferably nitrogen.

B is preferably CR¹.

In certain embodiments of the invention —W—X—Y— represents a chain of 2 to 6 atoms in length. —W—X—Y— preferably represents a 4 or 5 atom chain.

When W is C₂₋₃ alkenylene, the stereochemistry at the double bond is preferably (E).

X is preferably CH₂, O or NR⁵.

Exemplary G groups include, CHR³, N—C(O)OR⁴, N—C(O)NR⁴R⁵, N—C₁₋₄alkylene-C(O)OR⁴, N—C(O)C(O)OR⁴ and N-heteroaryl. G is preferably N—C(O)OR⁴, N—C(O)NR⁴R⁵, N—C₁₋₄alkylene-C(O)OR⁴, N—C(O)C(O)OR⁴, N-heterocyclyl, N-heteroaryl, N—S(O)₂R⁴, N—C(O)R⁴ or N—P(O)(O-Ph)₂; especially N—C(O)OR⁴, N—C(O)NR⁴R⁵, N—C₁₋₄alkylene-C(O)OR⁴, N-heteroaryl, N—S(O)₂R⁴ or N—C(O)R⁴; in particular N—C(O)OR⁴, N—C(O)NR⁴R⁵, N-heteroaryl, N—S(O)₂R⁴ or N—C(O)R⁴. More preferably, G is N—C(O)OR⁴, N—C(O)NR⁴R⁵ or N-heteroaryl. G is most preferably NCOOR⁴. When G is N-heteroaryl the heteroaryl ring is preferably pyrimidinyl or pyridinyl, especially pyrimidinyl e.g. pyrimidin-2-yl.

Exemplary R¹ groups include hydrogen, CN, halogen for example chloro or bromo, and C₁₋₄alkyl. R¹ is preferably chloro, C₁₋₄alkyl, hydrogen or cyano, for example R¹ is C₁₋₄ alkyl, hydrogen or cyano, especially methyl.

R² is preferably C₁₋₄alkyl. Exemplary R² groups include methyl.

Exemplary R³ groups include pentyl.

Exemplary R⁴ groups include methyl, ethyl, propyl, iso-propyl, sec-butyl, tert-butyl, butynyl, cyclobutyl, pentyl, 2,2-dimethylpropyl, cyclopentyl, hexyl, cyclohexyl, trifluoroethyl, trichloroethyl, phenyl, methoxyphenyl, tolyl, fluorophenyl, chlorophenyl, trifluoromethylphenyl, nitrophenyl, naphthalenyl, chlorobenzyl, methylsulfanylethyl- and tetrahydrofuranmethyl-.

Preferably R⁴ represents C₁₋₈ alkyl, C₂₋₈ alkenyl or C₂₋₈ alkynyl optionally substituted by one or more halo atoms or cyano, and may contain a CH₂ group that is replaced by O or S; or a C₃₋₇cycloalkyl, aryl or C₁₋₄alkylC₃₋₇ cycloalkyl, any of which may be substituted with one or more substituents selected from halo, C₁₋₄ alkyl, C₁₋₄ fluoroalkyl, OR⁵, CN, NR⁵R⁵⁵, NO₂ or C(O)OC₁₋₄alkyl. More preferably R⁴ represents C₁₋₈ alkyl, C₂₋₈ alkenyl or C₂₋₈ alkynyl optionally substituted by one or more halo atoms or cyano, and may contain a CH₂ group that is replaced by O or S; or a C₃₋₇cycloalkyl or aryl, either of which may be substituted with one or more substituents selected from halo, C₁₋₄ alkyl, C₁₋₄ fluoroalkyl, OR⁵, CN, NR⁵R⁵⁵, NO₂ or C(O)OC₁₋₄alkyl. Most preferred R⁴ groups are C₃₋₅alkyl optionally substituted by one or more halo atoms or cyano, which may contain a CH₂ group that is replaced by O or S, or C₃₋₅cycloalkyl optionally substituted by C₁₋₄ alkyl. In one embodiment of the invention the group represented by R⁴ is unsubstituted.

Exemplary R⁵ groups include hydrogen and methyl.

Preferably R⁶ is hydrogen, halogen, CN, C₁₋₄alkyl, C₁₋₄alkoxy, ethynyl or C₁₋₄alkyleneS(O)_{f}. More preferably R⁶ is hydrogen, methyl or halogen, especially hydrogen or methyl.

In one embodiment of the invention d+e is 2, 3, or 4. In a preferred embodiment of the invention d and e each represent 1. In a more preferred embodiment of the invention d and e each represent 2.

Examples of independent R⁷ and R⁷⁷ groups include hydrogen and methyl, especially hydrogen. An example of a heterocyclic group where R⁷ and R⁷⁷ are taken together is piperidine.

R⁸ is preferably hydrogen or halogen e.g. fluoro. In one embodiment R⁸ is hydrogen.

Preferably R¹¹ represents hydrogen.

When B represents nitrogen, suitably X does not represent C(O)NR².

When —W—X—Y— represents —NHC₀₋₄alkyl-, suitably R⁴ is C₁₋₈ alkyl, C₂₋₈ alkenyl or C₂₋₈ alkynyl, any of which may be optionally substituted by one or more halo atoms, NR⁵R⁵⁵, OR⁵, C(O)OR⁵, OC(O)R⁵ or cyano, and which may contain a CH₂ group that is replaced by O or S; or a C₃₋₇cycloalkyl, heterocyclyl, C₁₋₄alkyleneC₃₋₇cycloalkyl or C₁₋₄alkyleneheterocyclyl, any of which may be substituted with one or more substituents selected from halo, C₁₋₄ alkyl, C₁₋₄ fluoroalkyl, OR⁵, CN, NR⁵R⁵⁵, SO₂Me, NO₂ or C(O)OR⁵.

When X represents C(O)NR², preferably W is not a bond or alternatively Y is a bond or contains at least two carbon atoms linking X to the heterocyclic ring.

While the preferred groups for each variable have generally been listed above separately for each variable, preferred compounds of this invention include those in which several or each variable in formula (I) is selected from the preferred, more preferred or particularly listed groups for each variable. Therefore, this invention is intended to include all combinations of preferred, more preferred and particularly listed groups.

Specific compounds of the invention which may be mentioned are those included in the Examples and pharmaceutically acceptable salts thereof.

As used herein, unless stated otherwise, “alkyl” as well as other groups having the prefix “alk” such as, for example, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃.

The term “cycloalkyl” means carbocycles containing no heteroatoms, and includes monocyclic and bicyclic saturated and partially saturated carbocycles. Examples of cycloalkyl include cyclopropyl, cyclobutyl, cyclopentyl, cyclohexyl and cycloheptyl. Examples of partially saturated cycloalkyl groups include cyclohexene and indane. Cycloalkyl groups will typically contain 3 to 10 ring carbon atoms in total (e.g. 3 to 6, or 8 to 10).

The term “halo” includes fluorine, chlorine, bromine, and iodine atoms.

The term “aryl” includes phenyl and naphthyl, in particular phenyl.

Unless otherwise indicated the term “heterocyclyl” and “heterocyclic ring” includes 4- to 10-membered monocyclic and bicyclic saturated rings, e.g. 4- to 7-membered monocyclic saturated rings, containing up to three heteroatoms selected from N, O and S. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes mono- and bicyclic 5- to 10-membered, e.g. monocyclic 5- or 6-membered, heteroaryl rings containing up to 4 heteroatoms selected from N, O and S. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl and triazinyl. Bicyclic heteroaryl groups include bicyclic heteroaromatic groups where a 5- or 6-membered heteroaryl ring is fused to a phenyl or another heteroaromatic group. Examples of such bicyclic heteroaromatic rings are benzofuran, benzothiophene, indole, benzoxazole, benzothiazole, indazole, benzimidazole, benzotriazole, quinoline, isoquinoline, quinazoline, quinoxaline and purine.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts thereof, and mixtures thereof, except where specifically drawn or stated otherwise.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like Since the compounds of formula (I) are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, in which R⁶, R⁸, A, B, d, e, W, X, Y and G are as defined above and which are illustrated in the schemes below for compounds where R¹¹ is hydrogen.

Compounds of formula (I) in which X is CO₂, COS, or CONR² can be prepared by condensing the appropriate acid (II) with an alcohol, thiol, or amine (III), as shown in Scheme 1 where E is O, S, or NR², using a typical reagent for such a condensation reaction, e.g., EDCI (Pottorf, R. S.; Szeto, P. In Handbook of Reagents for Organic Synthesis: Activating Agents and Protecting Groups; Pearson, A. J., Roush, W. R., Eds.; Wiley: Chichester, 1999; pp 186-188). The acids (II) and alcohols, thiols, and amines (III) are either commercially available or are prepared easily using known techniques.

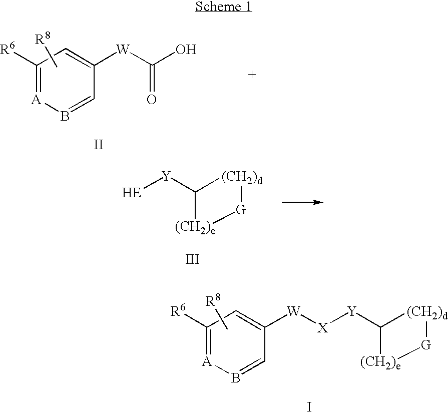

Compounds of formula (I) in which X is SCO or OCO can be prepared by condensing the appropriate thiol or alcohol (IV) with the appropriate acid (V), as shown in Scheme 2 where E is S or O, employing a reagent typically used for effecting such reactions, e.g., EDCI (Pottorf, R. S.; Szeto, P. In Handbook of Reagents for Organic Synthesis: Activating Agents and Protecting Groups; Pearson, A. J., Roush, W. R., Eds.; Wiley: Chichester, 1999; pp 186-188). The alcohols and thiols (IV), as well as acids (V), are either commercially available or are prepared straightforwardly using known techniques.

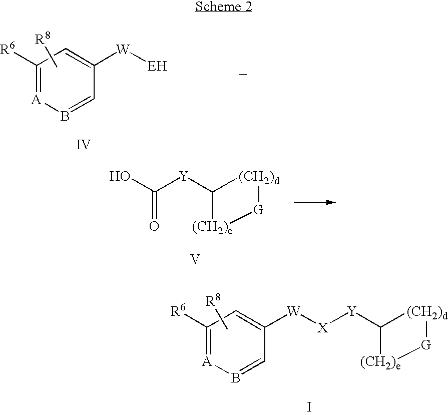

Compounds of formula (I) in which X is S or O can be prepared by alkylating the appropriate thiol or alcohol (IV) with the appropriate alkyl halide or sulfonate ester (VI), as shown in Scheme 3 where E is S or O and LG is chloro, bromo, iodo, alkanesulfonate, or arenesulfonate. The reaction is typically carried out using a base, e.g., potassium tert-butoxide (Hall, S. E., et al. J. Med. Chem. 1989, 32, 974-984). The alcohols and thiols (IV), as well as the alkyl halides or sulfonates (VI), are either commercially available or are made easily using known techniques. The compounds of formula (I) where X is SO or SO₂ can easily be obtained from the compounds of formula (I) where X is S by oxidation with, for example, mCPBA (Fyfe, M. C. T. et al. International Patent Publication WO 04/72031).

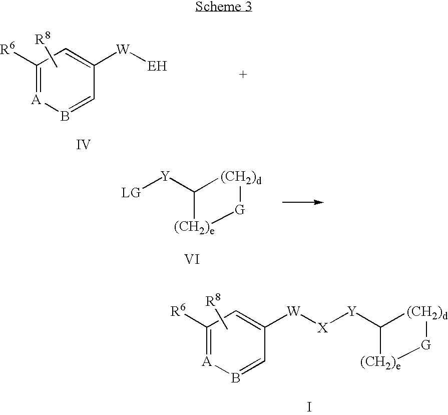

Compounds of formula (I) in which W is C₂₋₃ alkenylene can be prepared by a Wittig reaction between the appropriate phosphonium salt (VII) and the appropriate aldehyde (VIII), as indicated in Scheme 4 where m is 1 or 2 and n is 0 or 1 with the proviso that m+n<3. As an alternative, to the approach described in Scheme 4, the compounds of formula (I) in which W is C₂₋₃ alkenylene can be prepared by a Wittig reaction between the appropriate aldehyde (IX) and the appropriate phosphonium salt (X), as indicated in Scheme 5 where q is 0 or 1 and r is 1 or 2 with the proviso that q+r<3. The reactions are carried out in the presence of a suitable base, e.g., NaOMe or LiHMDS (March, J. Advanced Organic Chemistry, 4th edn.; Wiley: New York, 1992; pp 956-963). The phosphonium salts (VII) and (X), as well as the aldehydes (VIII) and (IX), are either commercially available or are made easily using known techniques. The compounds of formula (I) where W is C₂₋₃ alkylene can easily be synthesized from the compounds of formula (I) where W is C₂₋₃ alkenylene by a hydrogenation reaction using, for example, palladium on charcoal as a catalyst.

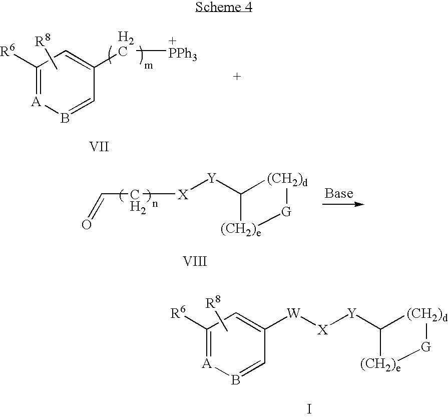

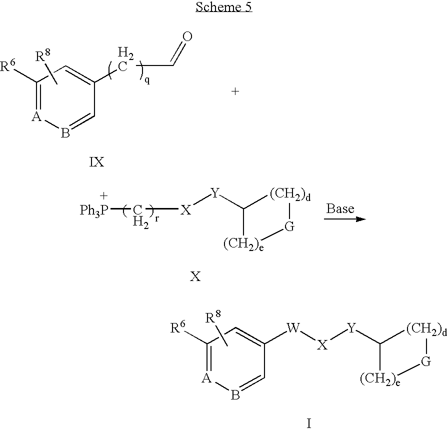

Compounds of the formula (I) where W is a bond, X is S or O, and A is N or C—R¹ where R¹ is CN can be prepared by condensation of the appropriate heteroaryl halide (XI), where with the appropriate alcohol or thiol (III), as depicted in Scheme 6 where Hal represents a halogen and E is S or O. The reaction is carried out in the presence of a suitable basic system, e.g., potassium hydroxide and potassium carbonate in the presence of tris(3,6-dioxaheptyl)amine (Ballesteros, P.; Claramunt, R. M.; Elguero, J. Tetrahedron 1987, 43, 2557-2564). The heteroaryl halides (XI) and alcohols/thiols (III) are either commercially available or are made easily using known techniques.

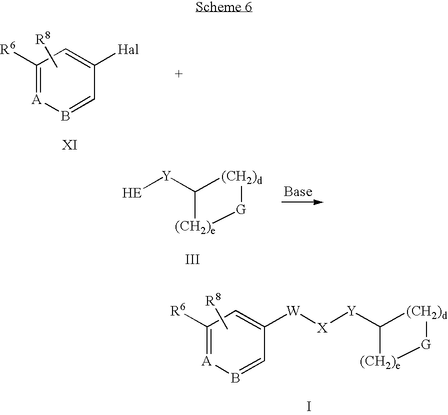

Compounds of the formula (I) where G is NC(O)OR⁴, NC(O)NR⁴R⁵, NC(O)R⁴, or N—C(O)C(O)OR⁴ can be prepared by the route shown in Scheme 7, where anamine of formula (XII) is condensed with an acyl chloride of formula (XIII) where A is O, NR⁵, a bond, or C(O)O. The reaction is carried out in the presence of a suitable base, such as triethylamine (Picard, F., et al. J. Med. Chem. 2002, 45, 3406-3417). Compounds of the formula (I) where G is NCONR⁴R⁵ and R⁵ is hydrogen may also be prepared by reacting the amine (XII) with a suitable isocyanate O═C═N—R⁴ (Boswell, R. F., Jr., et al. J. Med. Chem. 1974, 17, 1000-1008). Compounds of the formula (I) where G is N—C₁₋₄alkylene-C(O)OR⁴ may be prepared by alkylating the amine (XII) with the appropriate α-haloester (Rooney, C. S. et al. J. Med. Chem. 1983, 26, 700-714). The amine (XII) is generally derived from its N-tert-butoxycarbonyl precursor—prepared by one of the routes outlined in Schemes 1-6—by deprotection with an acid, e.g., trifluoroacetic acid (Fyfe, M. C. T. et al. International Patent Publication WO 04/72031).

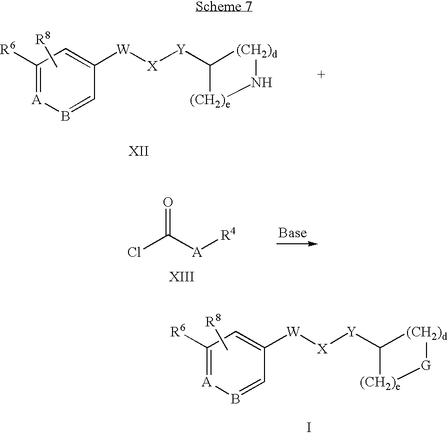

Compounds of the formula (I) where G is N-heteroaryl may be prepared by condensation of amine (XII) with a heteroaryl chloride of formula (XIV), as illustrated in Scheme 8 (Barillari, C. et al. Eur. J. Org. Chem. 2001, 4737-4741; Birch, A. M. et al. J. Med. Chem. 1999, 42, 3342-3355).

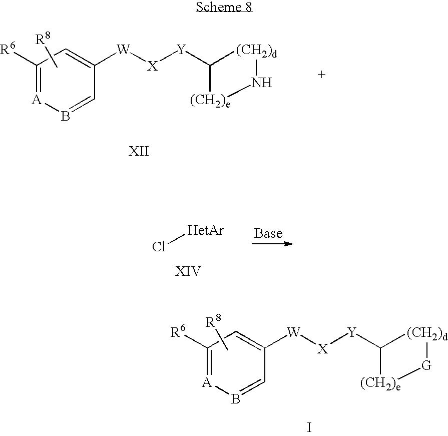

Compounds of the formula (I) where R¹ is CN can be prepared from the corresponding unsubstituted pyridine by the Reissert reaction (Fife, W. K. J. Org. Chem. 1983, 48, 1375-1377). Similar reactions can be used to prepare the compounds where R¹ is a halogen (Walters, M. A.; Shay, J. J. Tetrahedron Lett. 1995, 36, 7575-7578). The compounds where R¹ is halogen can be transformed into the corresponding compounds where R¹ is C₁₋₄ alkyl by transition metal-catalysed cross-coupling reactions (Fërstner, A., et al. J. Am. Chem. Soc. 2002, 124, 13856-13863).

Other compounds of formula (I) may be prepared by methods analogous to those described above or by methods known per se.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

Any novel intermediates, such as those defined above, may be of use in the synthesis of compounds of formula (I) and are therefore also included within the scope of the invention, for example compounds of formula (XII):

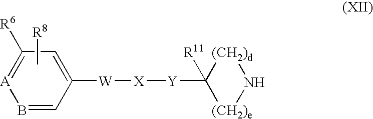

wherein the groups A, B, W, X, Y, d, e, R⁶ and R⁸ are as defined above for compounds of formula (I).

A further embodiment of the invention relates to compounds of formula (XII) wherein the groups R⁶ and R⁸ represent hydrogen, d and e each represent 2 and the groups A, B, W, X and Y are as defined above for compounds of formula (Ia).

As indicated above the compounds of formula (I) are useful as GPR116 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR116, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 10 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR116 plays a role.

Thus the invention also provides a method for the treatment of a disease or condition in which GPR116 plays a role comprising a step of administering to a subject in need thereof an effective amount of a compound of formula (I), or a pharmaceutically acceptable salt thereof. Diseases or conditions in which GPR116 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including Type 1 and Type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof, for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR116 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)-(−)-3-(4-chlorophenyl)-N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR116 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O—CH₃CN solution containing 0.1% HCO₂H over 6 min with UV detection at 220 nm. Gradient information: 0.0-0.3 min 100% H₂O; 0.3-4.25 min: Ramp up to 10% H₂O-90% CH₃CN; 4.25-4.4 min: Ramp up to 100% CH₃CN; 4.4-4.9 min: Hold at 100% CH₃CN; 4.9-6.0 min: Return to 100% H₂O. The mass spectra were obtained using an electrospray ionisation source in either the positive (ES⁺) or negative (ES⁻) ion modes. Jones reagent was prepared according to the method described by Meinwald J., et al. Org Synthesis, Coll. Vol. V, 866.

Abbreviations and acronyms: Ac: Acetyl; Boc: tert-Butoxycarbonyl; t-Bu: tert-Butyl; 18C6: 18-Crown-6; DIPEA: N,N-Diisopropylethylamine; DMAP: 4-(Dimethylamino)pyridine; DMF: N,N-Dimethylformamide; EDCI: 1-(3-Dimethylaminopropyl)-3-ethylcarbodiimide hydrochloride; Et: Ethyl; HOBt: 1-Hydroxybenzotriazole; IH: Isohexane; mCPBA: 3-Chloroperoxybenzoic acid; Me: Methyl; Ph: Phenyl; RP-HPLC: Reverse phase-high performance liquid chromatography; RT: Retention time; TFA: Trifluoroacetic acid; THF: Tetrahydrofuran. The syntheses of the following compounds have been described elsewhere: 4-(3-Bromo-2-oxopropyl)piperidine-1-carboxylic acid tert-butyl ester: Piotrowski, D. W., et al. WO 04/013137; 4-(3-Bromopropyl)pyridine hydrobromide: Elpern R., et al., J. Am. Chem. Soc. 1957, 79, 1951-1954; 3-(2-Cyanopyridin-4-yl)propyl acetate: Ornstein, P. L., et al. J. Med. Chem. 1991, 34, 90-97; 4-Mercaptopiperidine-1-carboxylic acid tert-butyl ester: Bru-Magniez, N., et al. U.S. Pat. No. 5,317,025; 4-(2-Methanesulfonyloxyethyl)piperidine-1-carboxylic acid tert-butyl ester: Cain, G. A., et al. U.S. Pat. No. 5,252,586; 4-Methylaminomethylpiperidine-1-carboxylic acid tert-butyl ester: Hiscock, S. D., et al. WO 03/049737; 4-(2-oxopropyl)piperidine-1-carboxylic acid tert-butyl ester: Piotrowski, D. W., et al. WO 04/013137; 4-(3-Oxopropyl)piperidine-1-carboxylic acid tert-butyl ester: Keenan, R. M., et al. J. Med. Chem. 1999, 42, 545-559; 4-Pentylcyclohexanecarboxylic acid amide: Obikawa, T.; Ikukawa, S., JP 03133963; Pyridin-4-ylmethylphosphonic acid diethyl ester: Hutchison A. J. et al., J. Med. Chem., 1989, 32, 2171-2178; 2-(4-Pyridyl)ethanethiol: Hayashi, H.; Hayashi, K., U.S. Pat. No. 6,720,426; 3-Pyridin-4-ylpropane-1-thiol: Burgess, D. M.; Bayer, H. O., J. Org. Chem. 1963, 28, 2283-2288; Triphenylpyridin-4-ylmethylphosphonium bromide: Carsky, P., et al. Liebigs Ann. 1980, 291-304. All other compounds were available from commercial sources.

### Preparation 1: 4-Mercaptomethylpiperidine-1-carboxylic acid tert-butyl ester

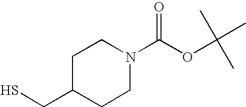

A stirred solution of N-tert-butoxycarbonyl-4-(4-toluenesulfonyloxymethyl)piperidine (240 mg, 0.65 mmol) and thiourea (99 mg, 1.30 mmol) in EtOH (1 mL) was heated under gentle reflux for 16 h. The solvent was evaporated off under reduced pressure to furnish the tosylate salt of 4-carbamimidoylsulfanylmethylpiperidine-1-carboxylic acid tert-butyl ester: m/z (ES⁺)=274.0 [M+H]⁺. A solution of this salt (250 mg, 0.56 mmol) in H₂O (1 mL) and concentrated aqueous NH₃ (2 mL) was heated to 100° C. with stirring for 20 min. On cooling, the mixture was partitioned between Et₂O (30 mL) and H₂O (10 mL). The pH of the aqueous phase was adjusted to 7 using 2 M HCl and saturated aqueous NaHCO₃. The organic phase was extracted with 1 M NaOH (15 mL), then the aqueous extracts were neutralised to pH 7 with 2 M HCl. The cloudy mixture was extracted with Et₂O (50 mL), then the organic extracts were washed with brine (10 mL) and dried (MgSO₄). Filtration and solvent evaporation furnished the title compound: δ_{H} (CDCl₃) 1.05-1.20 (m, 2H), 1.35 (t, 1H), 1.48 (s, 9H), 1.50-1.60 (m, 1H), 1.80-1.90 (m, 2H), 2.40-2.50 (m, 2H), 2.60-2.80 (m, 2H), 4.05-4.25 (m, 2H).

### Preparation 2: 4-(2-Methylaminoethyl)piperidine-1-carboxylic acid tert-butyl ester

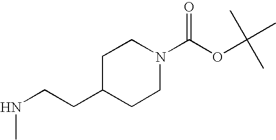

MeNH₂ (1.41 mL of a 2.0 M solution in THF, 2.82 mmol) was added to a stirred solution of 4-(2-oxoethyl)piperidine-1-carboxylic acid tert-butyl ester (639 mg, 2.81 mmol) in anhydrous PhMe (2 mL). After 30 min, the solution was concentrated in vacuo, then anhydrous THF (2 mL) and anhydrous MeOH (2 mL) were added. The stirred solution was treated with NaBH₄ (128 mg, 3.38 mmol). After 3 h, the solvents were removed under reduced pressure and the residue partitioned between EtOAc (25 mL) and H₂O (20 mL). The organic layer was washed with brine (20 mL), dried (MgSO₄), filtered, and concentrated to furnish the title compound: δ_{H} (CDCl₃) 1.00-1.10 (m, 2H), 1.35-1.45 (m, 10H), 1.50-1.65 (m, 4H), 2.39 (s, 3H), 2.50-2.70 (m, 4H), 3.90-4.10 (m, 2H).

### Preparation 3: 4-(3-Hydroxypropyl)pyridine-2-carbonitrile

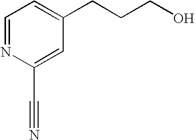

A solution of K₂CO₃ (1.67 g, 12.1 mmol) in H₂O (30 mL) was added to a stirred solution of 3-(2-cyanopyridin-4-yl)propyl acetate (4.94 g, 24.2 mmol) in MeOH (130 mL). After 25 min, the MeOH was removed under reduced pressure, then the aqueous phase was extracted three times with EtOAc. The combined organic extracts were dried (MgSO₄), filtered, and concentrated to give a residue that was purified by column chromatography (IH-EtOAc, 1:3) to furnish the title compound: m/z (ES⁺)=163.1 [M+H]⁺.

### Preparation 4: [3-(1-tert-Butoxycarbonylpiperidin-4-yl)-2-oxopropyl]triphenylphosphonium bromide

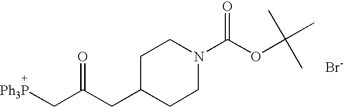

A solution of 4-(3-bromo-2-oxopropyl)piperidine-1-carboxylic acid tert-butyl ester (320 mg, 1.0 mmol) and PPh₃ (267 mg, 1.0 mmol) in anhydrous THF (10 mL) was heated under reflux with stirring for 3 h. The solvent was removed under reduced pressure, then the residue was dissolved in a MeCN-PhMe solution. The solvents were evaporated off in vacuo to give a solid that was triturated with Et₂O. The solid was filtered and washed with more Et₂O to furnish the title compound: δ_{H} (CD₃CN) 1.00-1.10 (m, 2H), 1.20-1.30 (m, 1H), 1.46 (s, 9H), 1.50-1.57 (m, 2H), 2.65-2.80 (m, 4H), 3.95-4.05 (m, 2H), 5.03 (d, 2H), 7.70-7.80 (m, 12H), 7.85-7.95 (m, 3H).

### Preparation 5: 4-(4-Piperidin-4-ylbutyl)pyridine

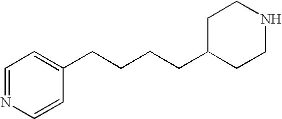

TFA (10 mL) was added over 2 min to a stirred solution of 4-(4-pyridin-4-ylbutyl)piperidine-1-carboxylic acid tert-butyl ester (Example 53, 1.13 g, 3.6 mmol) in CH₂Cl₂ (15 mL). After 1 h at 20° C., the mixture was concentrated under reduced pressure and the residue was dissolved in EtOAc (180 mL). The solution was washed with 1 M NaOH (30 mL) and brine (30 mL), before being dried (MgSO₄), filtered, and evaporated. The residue was taken up in EtOAc (5 mL) and filtered through celite. Solvent evaporation furnished the title compound: m/z (ES⁺)=219.2 [M+H]⁺.

### Preparation 6: tert-Butylmethylcarbamoyl chloride

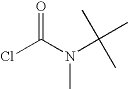

Anhydrous pyridine (162 μL, 2.0 mmol) was added to a vigorously stirred solution of triphosgene (208 mg, 0.7 mmol) in anhydrous CH₂Cl₂ (5 mL). tert-Butylmethylamine (240 μL, 2.0 mmol) was added, then the mixture was stirred at 20° C. for 3 d. The CH₂Cl₂ was removed at 20° C./230 mmHg, then Et₂O (20 mL) was added. The precipitated solids were removed by filtration, then the Et₂O was evaporated at 20° C./250 mmHg to furnish the title compound: δ_{H} (CDCl₃) 1.45 (s, 9H), 3.15 (s, 3H).

The compounds listed in Table 1 were prepared from the corresponding alcohols using methods similar to that described in Example 59

TABLE 1

| | | | RT | m/z |
|---|---|---|---|---|
| Prep | Structure | Name | (min) | (ES⁺) |
| 7 | 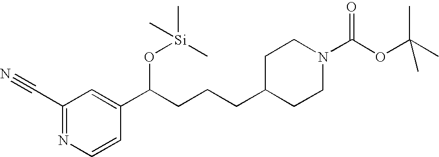 | 4-[4-(2-Cyanopyridin-4-yl)-4-trimethylsilanyloxybutyl]piperidine-1-carboxylic acidtert-butyl ester | 4.61 | 432.4[M + H]⁺ |
| 8 | 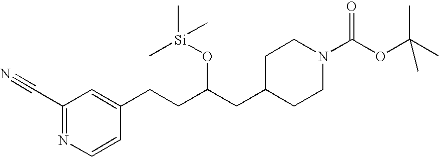 | 4-[4-(2-Canopyridin-4-yl)-2-trimethylsilanyloxybutyl]piperidine-1-carboxylic acidtert-butyl ester | 4.82 | 432.2[M + H]⁺ |
| 9 | 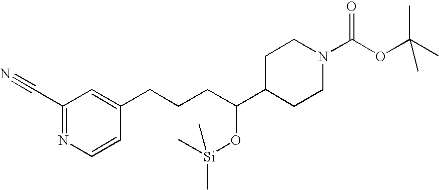 | 4-[4-(2-Cyanopyridin-4-yl)-1-trimethylsilanyloxybutyl]piperidine-1-carboxylic acidtert-butyl ester | 4.42 | 432.2[M + H]⁺ |

### Preparation 10: (2-Methylpyridin-4-ylmethyl)triphenylphosphonium chloride

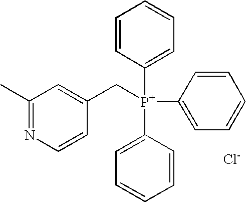

Thionyl chloride (13.64 mL, 187 mmol) was added slowly over 10 min to an ice-cooled, stirred solution of (2-methylpyridin-4-yl)methanol (1.314 g, 10.68 mmol) in dry CH₂Cl₂ (60 mL). After warming to room temperature and stirring for a further 1.5 h, toluene (50 mL) was added followed by sufficient saturated aqueous Na₂CO₃ to achieve pH 10 in the aqueous phase. The aqueous component was separated and extracted further with toluene (2×30 mL). The combined organic phases were dried (MgSO₄), sodium iodide (200 mg) and triphenylphosphine (8.40 g, 32.0 mmol) were added, and the solvent volume reduced to 10 mL. The stirred mixture was heated at 60° C. for 48 h, cooled and the solid collected by filtration. This was washed with ether (10 mL) and air-dried to give the title phosphonium salt: δ_{H} (DMSO) 2.26 (s, 3H), 5.20 (d, 2H), 6.70 (s, 1H), 6.82 (d, 1H), 7.70-7.79 (m, 12H), 7.91-7.94 (m, 3H), 8.30 (d, 1H).

### Preparation 11: 2-Hydroxy-1-oxa-8-azaspiro[4.5]decane-8-carboxylic acid tert-butyl ester

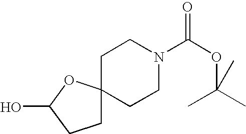

A stirred solution of 4-hydroxy-4-(3-hydroxypropyl)piperidine-1-carboxylic acid tert-butyl ester (1.0 g, 3.86 mmol) in CH₂Cl₂ (60 mL) was cooled on an ice bath and Dess-Martin periodinane (1.8 g, 4.24 mmol) added. After 1 h, the reaction mixture was diluted with ether (120 mL) and washed with 2 M aqueous NaOH (70 mL). The aqueous phase was extracted with ether (60 mL) and the combined organic phases washed with water (50 mL) and brine (50 mL) then dried (MgSO₄). The solvent was removed and the residue purified by column chromatography (IH-EtOAc 3:2) to afford the title compound: δ_{H} (CDCl₃) 1.48 (s, 9H), 1.48-1.62 (m, 2H), 1.62-1.77 (m, 3H), 1.91-2.08 (m, 3H), 3.35 (m, 2H), 3.56 (m, 2H), 5.51 (d, 1H).

### Example 1

### 4-(3-Pyridin-4-ylpropylsulfanylcarbonyl)piperidine-1-carboxylic acid tert-butyl ester

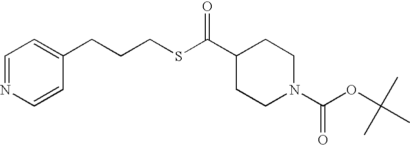

A mixture of 1-(tert-butoxycarbonyl)isonipecotic acid (90 mg, 390 μmol) and EDCI (94 mg, 490 μmol) in anhydrous CH₂Cl₂ (3 mL) was stirred for 30 min, before being treated with a solution of DMAP (8 mg, 65 μmol) and 3-pyridin-4-ylpropane-1-thiol (50 mg, 326 μmol) in anhydrous CH₂Cl₂ (1 mL). After 20 h, the reaction mixture was concentrated to ca. 1 mL, then Et₂O (2 mL) was added. The solvent was decanted from the resulting gum which was chromatographed (Et₂O) to provide the title compound: RT=3.49 min; m/z (ES⁺)=365.0 [M+H]⁺.

The compounds shown in Table 2 were prepared by condensation of a thiol or alcohol with the appropriate acid and employed protocols similar to those described in Example 1.

TABLE 2

| | | | Rt | |
|---|---|---|---|---|
| Eg | Structure | Name | (min) | m/z (ES⁺) |
| 2 | 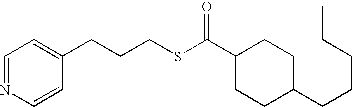 | 4-Pentylcyclohexanecarbothionic acid S-(3-pyridin-4-ylpropyl)ester | 4.52 | 334.0[M + H]⁺ |
| 3 | 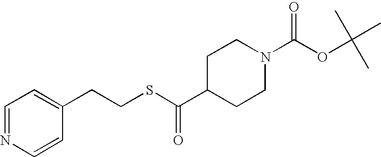 | 4-(2-Pyridin-4-ylethylsulfanylcarbonyl)piperidine-1-carboxylic acid tert-butyl ester | 2.87 | 351.2[M + H]⁺ |
| 4 | 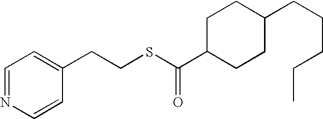 | 4-Pentylcyclohexanecarbothioic acid S-(2-pyridin-4-ylethyl)ester | 4.22 | 320.2[M + H]⁺ |
| 5 | 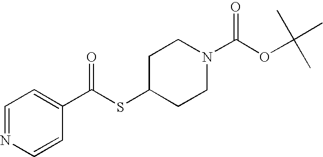 | 4-(Pyriidne-4-carbonylsulfanyl)piperidine-1-carboxylic acid tert-butyl ester | 3.84 | 323.0[M + H]⁺ |
| 6 | 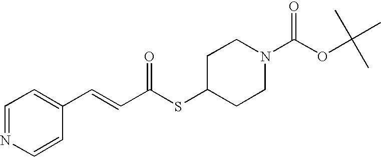 | (E)-4-(3-Pyridin-4-ylacryloylsulfanyl)piperidine-1-carboxylic acid tert-butylester | 3.61 | 349.0[M + H]⁺ |
| 7 | 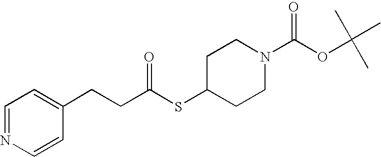 | 4-(3-Pyridin-4-ylpropionylsulfanyl)piperidine-1-carboxylic acid tert-butylester | 3.11 | 351.0[M + H]⁺ |
| 8 | 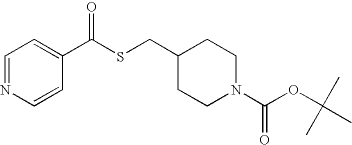 | 4-(Pyridine-4-carbonylsulfanylmethyl)piperidine-1-carboxylicacid tert-butyl ester | 3.92 | 321.9[M + 2H +MeCN-t-Bu]⁺ |
| 9 | 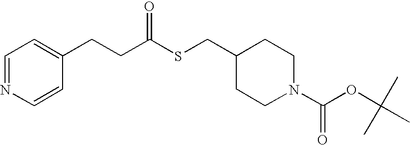 | 4-(3-Pyridin-4-ylpropionylsulfanylmethyl)piperidine-1-carboxylic acid tert-butyl ester | 3.99 | 365.0[M + H]⁺ |
| 10 | 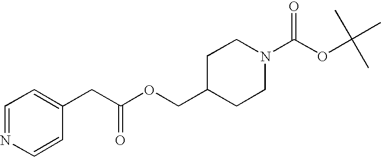 | 4-(2-Pyridin-4-ylacetoxymethyl)piperidine-1-carboxylic acid tert-butyl ester | 3.17 | 335.0[M + H]⁺ |
| 11 | 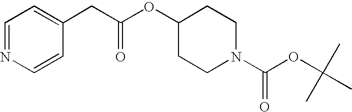 | 4-(2-Pyridin-4-ylacetoxy)piperidine-1-carboxylic acid tert-butyl ester | 3.15 | 321.0[M + H]⁺ |
| 12 | 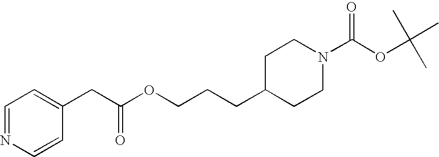 | 4-[3-(2-Pyridin-4-ylacetoxy)propyl]piperidine-1-carboxylic acid tert-butyl ester | 3.31 | 363.0[M + H]⁺ |
| 13 | 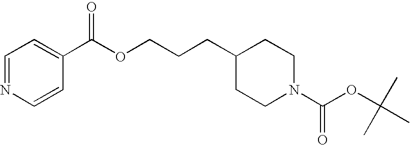 | Isonicotinic acid 3-(1-tert-butoxycarbonylpiperidin-4-yl)propyl ester | 4.32 | 349.0[M + H]⁺ |
| 14 | 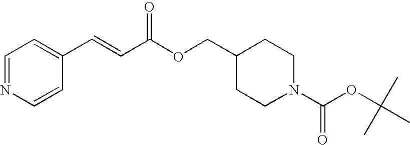 | (E)-4-(3-Pyridin-4-ylacryloyloxymethyl)piperidine-1-carboxylic acidtert-butyl ester | 3.56 | 347.0[M + H]⁺ |
| 15 | 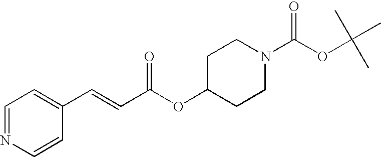 | (E)-4-(3-Pyridin-4-ylacryloyloxy)piperidine-1-carboxylic acid tert-butyl ester | 3.45 | 332.9[M + H]⁺ |
| 16 | 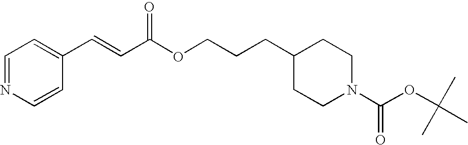 | (E)-4-[3-(3-Pyridin-4-ylacryloyloxy)propyl]piperidine-1-carboxylic acid tert-butylester | 3.94 | 375.0[M + H]⁺ |
| 17 | 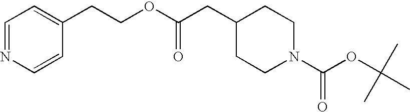 | 4-(2-Pyridin-4-ylethoxycarbonylmethyl)piperidine-1-carboxylic acid tert-butyl ester | 2.97 | 349.0[M + H]⁺ |
| 18 | 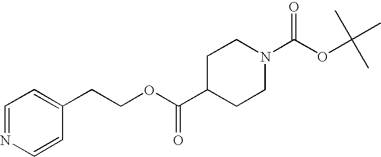 | Piperidine-1,4-dicarboxylicacid 1-tert-butyl ester 4-(2-pyridin-4-ylethyl)ester | 2.64 | 335.1[M + H]⁺ |
| 19 | 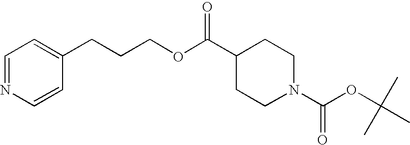 | Piperidine-1,4-dicarboxylicacid 1-tert-butyl ester 4-(3-pyridin-4-ylpropyl)ester | 2.67 | 349.1[M + H]⁺ |

### Example 20

### (E)-4-[Methyl(3-pyridin-4-ylacryloyl)amino]piperidine-1-carboxylic acid tert-butyl ester

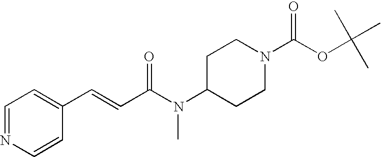

A mixture of (E)-3-pyridin-4-ylacrylic acid (100 mg, 671 μmol), EDCI (133 mg, 671 μmol), HOBt (91 mg, 671 μmol), NEt₃ (94 μL, 671 μmol), and anhydrous CH₂Cl₂ (3 mL) was stirred for 20 min, before being treated with 1-tert-butoxycarbonyl-4-methylaminopiperidine (131 mg, 610 μmol). After 3 d, the reaction mixture was diluted with CH₂Cl₂ (2 mL), before being washed with H₂O (10 mL), saturated aqueous NaHCO₃ (10 mL), and brine (10 mL). The CH₂Cl₂ solution was dried (MgSO₄), filtered, and concentrated. The residue was purified by RP-HPLC to furnish the title compound: RT=2.81 min; m/z (ES⁺)=346.2 [M+H]⁺.

The compounds shown in Table 3 were prepared by condensation of the appropriate acid with an amine utilising protocols similar to those described in Example 20.

TABLE 3

| | | | RT | m/z |
|---|---|---|---|---|
| Eg | Structure | Name | (min) | (ES⁺) |
| 21 | 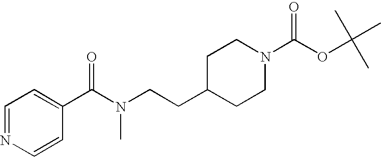 | 4-{2-[Methyl(pyridine-4-carbonyl)amino]ethyl}piperidine-1-carboxylic acid tert-butyl ester | 3.15 | 348.2[M +H]⁺ |
| 22 | 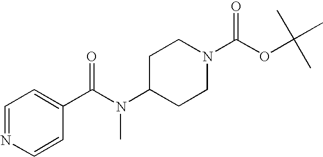 | 4-[Methyl(pyridine-4-carbonyl)amino]piperidine-1-carboxylic acid tert-butyl ester | 2.87 | 639.4[2M +H]⁺ |
| 23 | 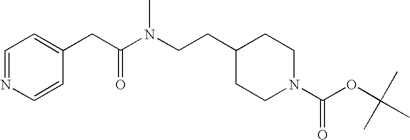 | 4-{2-[Methyl(2-pyridin-4-ylacetyl)amino]ethyl}piperidine-1-carboxylic acid tert-butyl ester | 2.72 | 362.2[M +H]⁺ |
| 24 | 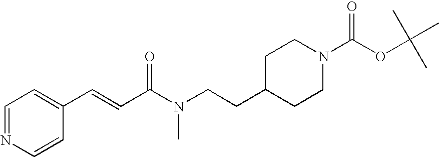 | 4-{2-[Methyl(3-pyridin-4-ylacryloyl)amino]ethyl}piperidine-1-carboxylic acid tert-butyl ester | 2.99 | 374.2[M +H]⁺ |
| 25 | 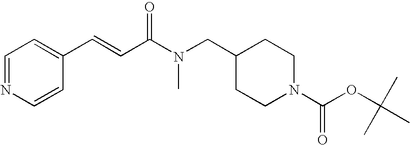 | 4-{[Methyl-(3-pyridin-4-ylacryloyl)amino]methyl}piperidine-1-carboxylic acid tert-butyl ester | 2.76 | 360.2[M +H]⁺ |

### Example 26

### 4-(2-Pyridin-4-ylethylsulfanylmethyl)piperidine-1-carboxylic acid tert-butyl ester

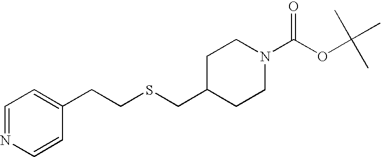

A stirred solution of 2-(4-pyridyl)ethanethiol (213 mg, 1.53 mmol) in anhydrous THF (2 mL) was treated with t-BuOK (63 mg, 0.56 mmol). 4-Methanesulfonyloxymethylpiperidine-1-carboxylic acid tert-butyl ester (150 mg, 0.51 mmol) was added, then the mixture was stirred at 20° C. for 1 h, before being heated under reflux. After 18 h, the reaction was cooled to 20° C., diluted with Et₂O (20 mL) and was washed with saturated aqueous NaHCO₃ (10 mL) and brine (10 mL), before being dried (MgSO₄). Filtration, solvent evaporation, and column chromatography (EtOAc) provided the title compound: RT=2.92 min; m/z (ES⁺)=337.1 [M+H]⁺.

The compounds catalogued in Table 4 were prepared using the t-BuOK-mediated alkylation of a pyridine-containing thiol or alcohol with the appropriate piperidine-containing mesylate, as illustrated by Example 26.

TABLE 4

| | | | RT | |
|---|---|---|---|---|
| Eg | Structure | Name | (min) | m/z (ES⁺) |
| 27 | 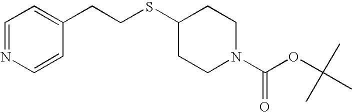 | 4-(2-Pyridin-4-ylethylsulfanyl)piperidine-1-carboxylic acid tert-butyl ester | 2.94 | 323.1[M + H]⁺ |
| 28 | 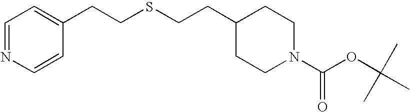 | 4-[2-(2-Pyridin-4-ylethylsulfanyl)ethyl]piperidine-1-carboxylic acid tert-butyl ester | 3.04 | 351.2[M + H]⁺ |
| 29 | 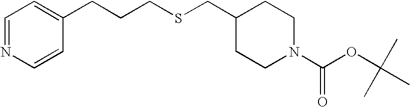 | 4-(3-Pyridin-4-ylpropylsulfanylmethyl)piperidine-1-carboxylic acid tert-butyl ester | 2.79 | 351.1[M + H]⁺ |
| 30 | 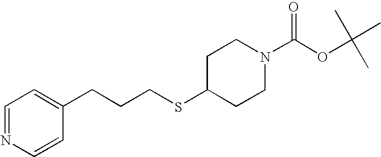 | 4-(3-Pyridin-4-ylpropylsulfanyl)piperidine-1-carboxylic acid tert-butyl ester | 2.84 | 337.2[M + H]⁺ |
| 31 | 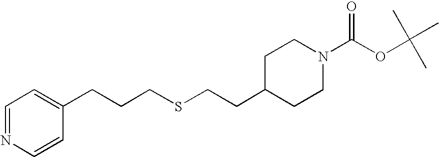 | 4-[2-(3-Pyridin-4-ylpropylsulfanyl)ethyl]piperidine-1-carboxylic acid tert-butyl ester | 2.89 | 365.2[M + H]⁺ |
| 32 | 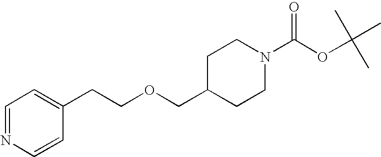 | 4-(2-Pyridin-4-ylethoxymethyl)piperidine-1-carboxylic acid tert-butyl ester | 2.72 | 321.2[M + H]⁺ |
| 33 | 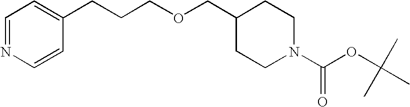 | 4-(3-Pyridin-4-ylpropoxymethyl)piperidine-1-carboxylic acid tert-butyl ester | 2.72 | 335.2[M + H]⁺ |
| 34 | 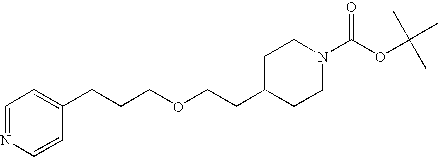 | 4-[2-(3-Pyridin-4-ylpropoxy)ethyl]piperidine-1-carboxylic acid tert-butyl ester | 2.76 | 349.2[M + H]⁺ |
| 35 | 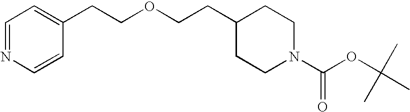 | 4-[2-(2-Pyridin-4-ylethoxy)ethyl]piperidine-1-carboxylic acid tert-butyl ester | 2.72 | 335.2[M + H]⁺ |
| 36 | 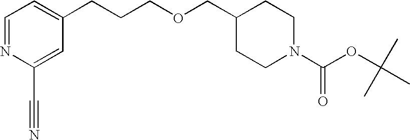 | 4-[3-(2-Cyanopyridin-4-yl)propoxymethyl]piperidine-1-carboxylic acid tert-butyl ester | 4.05 | 260.2[M − Boc +2H]⁺ |
| 37 | 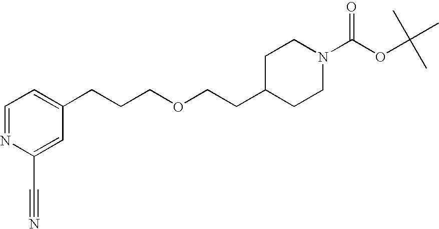 | 4-{2-[3-(2-Cyanopyridin-4-yl)propoxy]ethyl}piperidine-1-carboxylic acid tert-butyl ester | 4.12 | 274.2[M − Boc +2H]⁺ |
| 38 | 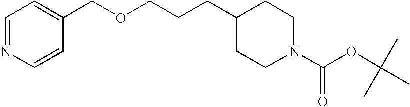 | 4-[3-(Pyridin-4-ylmethoxy)propyl]piperidine-1-carboxylic acid tert-butyl ester | 3.01 | 335.2[M + H]⁺ |
| 39 | 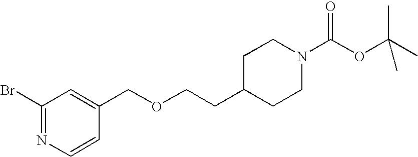 | 4-[2-(2-Bromopyridin-4-ylmethoxy)ethyl]piperidine-1-carboxylic acid tert-butyl ester | 3.99 | 399.0,401.0[M + H]⁺ |

### Example 40

### 4-(3-Pyridin-4-ylpropoxy)piperidine-1-carboxylic acid tert-butyl ester

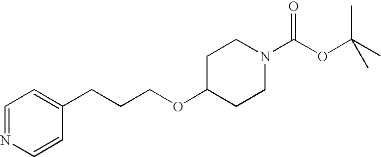

Solid t-BuOK 153 mg, 1.36 mmol) was added in one portion to a solution of 4-hydroxypiperidine-1-carboxylic acid tert-butyl ester (143 mg, 712 μmol) in anhydrous THF (6 mL) followed by 4-(3-bromopropyl)pyridine hydrobromide (200 mg, 712 μmol) and tetra-n-butylammonium iodide (26 mg, 71 μmol). After stirring for 24 h, the reaction mixture was diluted with EtOAc (50 mL) and washed with saturated aqueous NaHCO₃ (20 mL) and brine (20 mL) then dried (MgSO₄). Removal of the solvent and purification of the residue by column chromatography (IH-EtOAc, 5:1 to 4:1) afforded the title compound: RT=1.89 min; m/z (ES⁺)=321.3 [M+H]⁺.

### Example 41

### 4-[3-(Pyridin-4-yloxy)propyl]piperidine-1-carboxylic acid tert-butyl ester

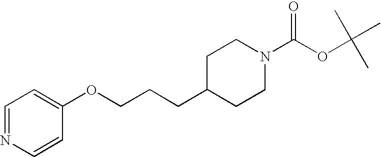

4-Chloropyridine hydrochloride (0.75 g, 5.0 mmol) was added to a stirred suspension of pulverised KOH (1.12 g, 20.0 mmol) and K₂CO₃ (0.69 g, 5.0 mmol) in anhydrous PhMe (50 mL). After 10 min, 4-(3-hydroxypropyl)piperidine-1-carboxylic acid tert-butyl ester (1.83 g, 7.5 mmol) and tris(3,6-dioxaheptyl)amine (160 μL, 0.5 mmol) were added, then the reaction heated to 120° C. for 20 h. On cooling to ambient temperature, the reaction mixture was washed with H₂O (2×15 mL) and brine (20 mL), before being dried (MgSO₄). Filtration, solvent evaporation, and column chromatography (IH-EtOAc, 7:3 to 1:4) provided the title compound: RT=2.82 min; m/z (ES⁺)=321.2 [M+H]⁺.

### Example 42

### 4-[2-(Pyridin-4-ylmethoxy)ethyl]piperidine-1-carboxylic acid tert-butyl ester

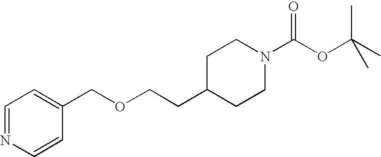

4-Chloromethylpyridine was reacted with 4-(2-hydroxyethyl)piperidine-1-carboxylic acid tert-butyl ester using similar conditions to those described in Example 41 to afford the title compound: RT=1.99 min; m/z (ES⁺)=321.3 [M+H]⁺.

### Example 43

### 4-(2-Oxo-2-pyridin-4-ylethylsulfanylmethyl)piperidine-1-carboxylic acid tert-butyl ester

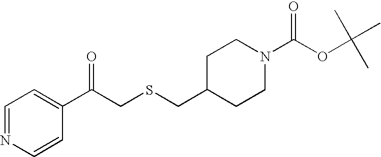

t-BuOK (44 mg, 400 μmol) was added to a stirred solution of 4-mercaptomethyl piperidine-1-carboxylic acid tert-butyl ester (Preparation 1, 50 mg, 216 μmol) and 2-bromo-1-pyridin-4-ylethanone hydrobromide (121 mg, 432 μmol) in anhydrous dioxane (3 mL). After 16 h, the reaction mixture was diluted with EtOAc (70 mL), before being washed with H₂O (15 mL) and brine (15 mL). After drying (MgSO₄), the organic phase was filtered, concentrated, and purified by column chromatography (IH-EtOAc, 1:1) to furnish the title compound: RT=3.72 min; m/z (ES⁺)=351.2 [M+H]⁺.

### Examples 44 and 45

### 4-(3-Pyridin-4-ylpropane-1-sulfonyl)piperidine-1-carboxylic acid tert-butyl ester and 4-(3-Pyridin-4-ylpropane-1-sulfinyl)piperidine-1-carboxylic acid tert-butyl ester

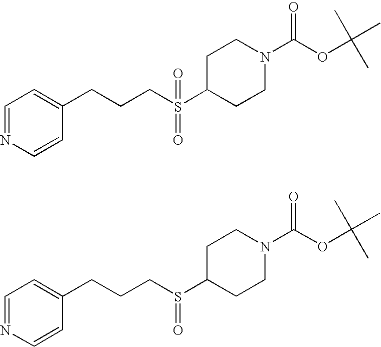

mCPBA (224 mg, 65% pure, 842 μmol) was added to a stirred solution of 4-(3-pyridin-4-ylpropylsulfanyl)piperidine-1-carboxylic acid tert-butyl ester (Example 30, 189 mg, 562 μmol) in CH₂Cl₂ (15 mL). After 2.5 h, the reaction was quenched with saturated aqueous Na₂CO₃ (5 mL). The organic layer was washed with brine, dried (MgSO₄), filtered, and concentrated, then the residue purified by column chromatography. The title sulfone eluted first using MeOH-EtOAc (1:19): RT=2.37 min; m/z (ES⁺)=369.1 [M+H]⁺. The title sulfoxide eluted with THF and was further purified by RP-HPLC: RT=2.32 min; m/z (ES⁺)=353.1 [M+H]⁺.

The compounds listed in Table 5 were prepared from the appropriate thioether employing the protocol exemplified by Examples 44 and 45.

TABLE 5

| | | | RT | m/z |
|---|---|---|---|---|
| Eg | Structure | Name | (min) | (ES⁺) |
| 46 | 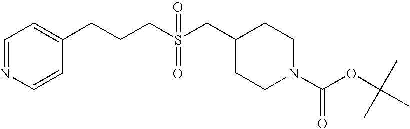 | 4-(3-Pyridin-4-ylpropane-1-sulfonylmethyl)piperidine-1-carboxylicacid tert-butyl ester | 2.42 | 383.1[M + H]⁺ |
| 47 | 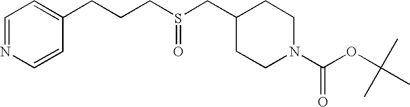 | 4-(3-Pyridin-4-ylpropane-1-sulfinylmethyl)piperidine-1-carboxylicacid tert-butyl ester | 2.36 | 367.2[M + H]⁺ |
| 48 | 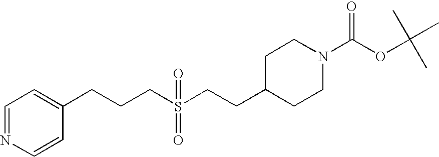 | 4-[2-(3-Pyridin-4-ylpropane-1-sulfonyl)ethyl]piperidine-1-carboxylicacid tert-butyl ester | 2.62 | 397.2[M + H]⁺ |
| 49 | 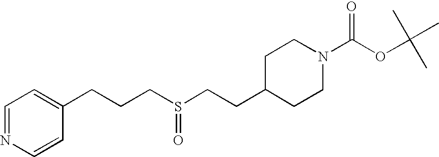 | 4-[2-(3-Pyridin-4-ylpropane-1-sulfinyl)ethyl]piperidine-1-carboxylicacid tert-butyl ester | 2.40 | 381.2[M + H]⁺ |
| 50 | 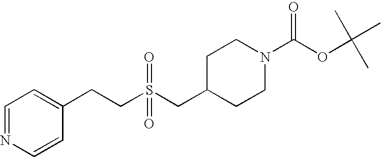 | 4-(2-Pyridin-4-ylethanesulfonylmethyl)piperidine-1-carboxylicacid tert-butyl ester | 2.62 | 369.2[M + H]⁺ |

### Example 51

### (E)-4-(2-Oxo-4-pyridin-4-ylbut-3-enyl)piperidine-1-carboxylic acid tert-butyl ester

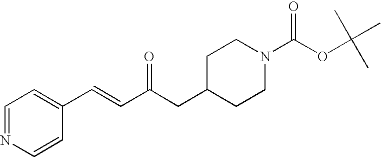

A solution of NaOMe (85 μL of a 25 wt % solution in MeOH, 372 μmol) in anhydrous MeOH (3 mL) was added dropwise over 25 min to a mixture of 4-pyridinecarboxaldehyde (43 mg, 401 μmol) and [3-(1-tert-butoxycarbonylpiperidin-4-yl)-2-oxopropyl]triphenylphosphonium bromide (Preparation 4, 200 mg, 343 μmol) in anhydrous DMF (7 mL). The mixture was stirred for 23.5 h, the solvents were evaporated under reduced pressure and the residue was partitioned between EtOAc (50 mL) and H₂O (25 mL). The aqueous phase was extracted with EtOAc (25 mL), then the combined organic extracts were washed with H₂O (20 mL) and dried (MgSO₄). Filtration, solvent evaporation, and purification by RP-HPLC afforded the title compound: RT=3.01 min; m/z (ES⁺)=331.2 [M+H]⁺.

### Examples 52 and 53

### (E)-4-(4-Pyridin-4-ylbut-3-enyl)piperidine-1-carboxylic acid tert-butyl ester and (Z)-4-(4-Pyridin-4-ylbut-3-enyl)piperidine-1-carboxylic acid tert-butyl ester

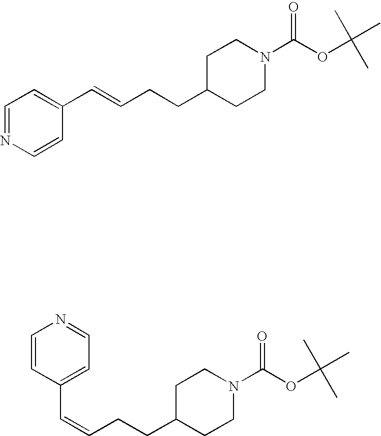

K₂CO₃ (145 mg, 1.05 mmol) and 18C6 (6 mg, 23 μmol) were added to a stirred solution of 4-(3-oxopropyl)piperidine-1-carboxylic acid tert-butyl ester (250 mg, 1.04 mmol) and triphenylpyridin-4-ylmethylphosphonium bromide (452 mg, 1.04 mmol) in anhydrous CH₂Cl₂ (8 mL). After 20 h, more K₂CO₃ (23 mg, 0.17 mmol) was added and stirring was continued for an additional 3 h. The mixture was partitioned between CH₂Cl₂ (25 mL) and H₂O (10 mL). The organic phase was washed with H₂O (10 mL), before being dried (MgSO₄). Filtration, solvent evaporation, and purification by RP-HPLC gave the title (E)-compound: δ_{H} (CDCl₃) 1.10-1.20 (m, 2H), 1.40-1.50 (m, 12H), 1.70-1.80 (m, 2H), 2.30-2.35 (m, 2H), 2.65-2.80 (m, 2H), 4.05-4.20 (br, 2H), 6.37 (d, 1H), 6.45-6.55 (m, 1H), 7.21 (d, 2H), 8.55 (d, 2H); RT=3.04 min; and (Z)-compound: δ_{H} (CDCl₃) 1.05-1.15 (m, 2H), 1.35-1.45 (m, 11H), 1.60-1.80 (m, 3H), 2.30-2.40 (m, 2H), 2.60-2.70 (m, 2H), 4.00-4.20 (br, 2H), 5.80-5.90 (m, 1H), 6.39 (d, 1H), 7.20 (d, 2H), 8.60 (d, 2H); RT=3.11 min.

The compounds listed in Table 6 were prepared via a Wittig reaction employing the protocol exemplified by Examples 52 and 53.

TABLE 6

| | | | RT | m/z |
|---|---|---|---|---|
| Eg | Structure | Name | (min) | (ES⁺) |
| 54 | 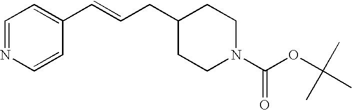 | (E)-4-(3-Pyridin-4-ylallyl)piperidine-1-carboxylic acid tert-butylester | 2.90 | 303.2[M + H]⁺ |
| 55 | 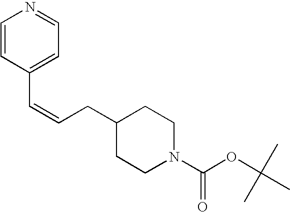 | (Z)-4-(3-Pyridin-4-ylallyl)piperidine-1-carboxylic acid tert-butylester | 2.84 | 303.2[M + H]⁺ |

### Example 56

### 4-(4-Pyridin-4-ylbutyl)piperidine-1-carboxylic acid tert-butyl ester

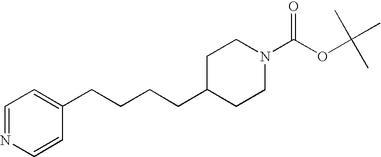

A stirred solution of (Z)-4-(4-pyridin-4-ylbut-3-enyl)piperidine-1-carboxylic acid tert-butyl ester (Example 53, 78 mg, 246 μmol) in EtOAc (4 mL) was treated with a slurry of Pd (30 mg, 10% on C, 28 μmol) in EtOAc (1 mL), before being placed under a H₂ atmosphere. After 4 h, the reaction mixture was filtered through a celite pad, washing with EtOAc (3×10 mL). The combined EtOAc solutions were concentrated to afford the title compound: RT=3.02 min; m/z (ES⁺)=319.3 [M+H]⁺.

### Example 57

### 4-(3-Pyridin-4-ylpropyl)piperidine-1-carboxylic acid tert-butyl ester

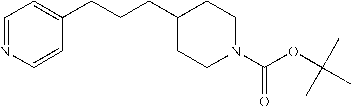

(Z)-4-(3-Pyridin-4-ylallyl)piperidine-1-carboxylic acid tert-butyl ester (Example 55) was reduced, utilising the protocol outlined above in Example 56, to produce the title compound: RT=2.86 min; m/z (ES⁺)=305.2 [M+H]⁺.

### Example 58

### 4-(2-Methyl-3-pyridin-4-ylpropyl)piperidine-1-carboxylic acid tert-butyl ester

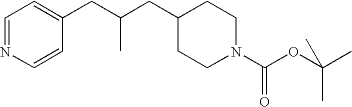

A solution of pyridin-4-ylmethylphosphonic acid diethyl ester (229 mg, 1 mmol) in anhydrous THF (4 mL) was treated with sodium hydride (40 mg of a 60% dispersion in oil, 1 mmol). After 10 min, a solution of 4-(2-oxopropyl)piperidine-1-carboxylic acid tert-butyl ester (241 mg, 1 mmol) in anhydrous THF (2 mL) was introduced and the reaction mixture stirred for 1 h at room temperature then at 70° C. for 3 h. On cooling, the reaction was poured into water (10 mL) and extracted with EtOAc (2×40 mL). The organic phase was washed with brine (20 mL), dried (MgSO₄) and evaporated. The residue was purified by column chromatography (1H-EtOAc, 2:1) to afford a mixture of (E)- and (Z)-(4-(2-methyl-3-pyridin-4-ylallyl)piperidine-1-carboxylic acid tert-butyl ester: RT=2.07 min; m/z (ES⁺)=317.4 [M+H]⁺. A solution of this mixture of olefins was reduced using the procedure described in Example 56 to give the title compound: RT=2.26 min; m/z (ES⁺)=319.4 [M+H]⁺.

### Example 59

### (E)-4-[4-(2-Cyanopyridin-4-yl)but-3-enyl]piperidine-1-carboxylic acid tert-butyl ester

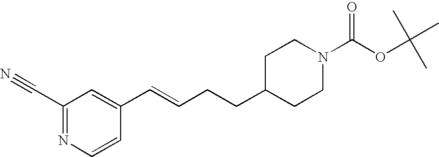

A solution of (E)-4-(4-pyridin-4-ylbut-3-enyl)piperidine-1-carboxylic acid tert-butyl ester (Example 52, 101 mg, 318 μmol) and mCPBA (78 mg, 70% pure, 318 μmol) in CH₂Cl₂ (4 mL) was stirred at 20° C. for 3 h. More mCPBA (12 mg, 70% pure, 49 μmol) was added, then the mixture was stirred for a further 45 min, before being purified by column chromatography (EtOAc then THF) to furnish (E)-4-[4-(1-oxypyridin-4-yl)but-3-enyl]piperidine-1-carboxylic acid tert-butyl ester: m/z (ES⁺)=333.2 [M+H]⁺. A mixture of this N-oxide (106 mg, 318 μmol), Me₃SiCN (170 μL, 1275 μmol) and NEt₃ (90 μL, 644 μmol) was heated at 100° C. for 1 h. On cooling to ambient temperature, the reaction mixture was diluted with EtOAc (20 mL) and washed with saturated aqueous NaHCO₃ (10 mL), H₂O (10 mL), and brine (10 mL). The organic phase was dried (MgSO₄), filtered, concentrated, and purified by column chromatography (IH-EtOAc, 9:1 to 4:1) to yield the title compound: RT=4.26 min; m/z (ES⁺)=683.5 [2M+H]⁺.

The compounds catalogued in Table 7 were prepared employing protocols similar to that described in Example 59.

TABLE 7

| | | | RT | |
|---|---|---|---|---|
| Eg | Structure | Name | (min) | m/z (ES⁺) |
| 60 | 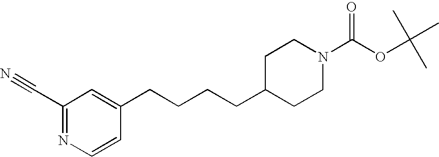 | 4-[4-(2-Cyanopyridin-4-yl)butyl]piperidine-1-carboxylic acid tert-butylester | 4.30 | 288.2[M + 2H-t-bu]⁺ |
| 61 | 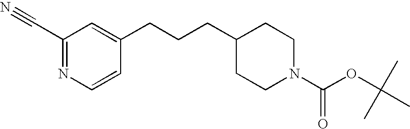 | 4-[3-(2-Cyanopyridin-4-yl)propyl]piperidine-1-carboxylic acid tert-butylester | 4.11 | 274.1[M + 2H-t-Bu]⁺ |
| 62 | 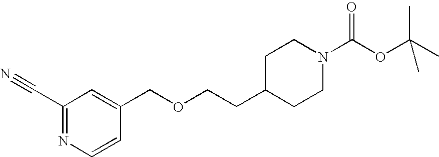 | 4-[2-(2-Cyanopyridin-4-ylmethoxy)ethyl]piperidine-1-carboxylic acid tert-butylester | 3.87 | 346.3 [M + H]⁺ |

### Example 63

### 4-(4-Pyridin-4-ylbutyl)piperidine-1-carboxylic acid tert-butylamide

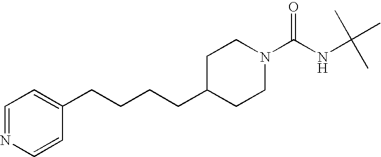

tert-Butyl isocyanate (9 μL, 75 μmol) was added to a stirred solution of 4-(4-piperidin-4-ylbutyl)pyridine (Preparation 5, 15 mg, 68 μmol) in anhydrous DMF (0.5 mL). After 18 h, the solvent was removed under reduced pressure, then the residue was dissolved in EtOAc (2 mL). The EtOAc solution was filtered through a short silica plug and evaporated to furnish the title compound: RT=2.64 min; m/z (ES⁺)=318.3 [M+H]⁺.

### Example 64

### 4-(4-Pyridin-4-ylbutyl)piperidine-1-carboxylic acid tert-butylmethylamide

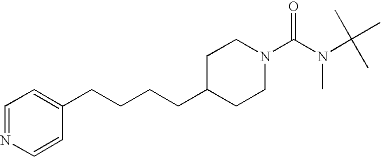

A solution of 4-(4-piperidin-4-ylbutyl)pyridine (Preparation 5, 40 mg, 183 μmol) in CH₂Cl₂ (1 mL) was added to tert-butylmethylcarbamoyl chloride (Preparation 6, 55 mg, 368 μmol). DIPEA (70 μL, 400 μmol) was added, then the mixture was stirred for 16 h at 20° C. The solvent was removed under reduced pressure, then the residue was purified twice by column chromatography (IH-EtOAc, 1:1) to give the title compound: RT=2.90 min; m/z (ES⁺)=332.3 [M+H]⁺.

### Example 65

### 4-(4-Pyridin-4-yl-butyl)piperidine-1-carboxylic acid 2,2,2-trichloroethyl ester

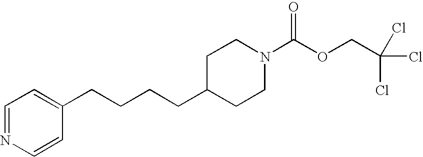

A solution of 2,2,2-trichloroethyl chloroformate (13 μL, 95 μmol) in anhydrous CH₂Cl₂ (0.2 ml) was added to a stirred solution of 4-(4-piperidin-4-ylbutyl)pyridine (Preparation 5, 22 mg, 100 μmol) and pyridine (8.5 μL, 105 μmol) in anhydrous CH₂Cl₂ (1 ml). After stirring at room temperature for 18 h, the reaction mixture was diluted with Et₂O (7 mL) and washed with 2 M aqueous NaOH (2 mL) and water (2 mL). The ethereal solution was then extracted with 1 M aqueous HCl (5 mL) and the aqueous phase basified to pH 9 using saturated aqueous Na₂CO₃. The resulting mixture was extracted with Et₂O (15 mL), which was then dried (MgSO₄) and evaporated to afford the title compound: RT=2.38 min; m/z (ES⁺)=393.2 [M+H]⁺.

The compounds listed in Table 8 were prepared from the appropriate chloroformates using methods similar to that described in Example 65.

TABLE 8

| | | | RT | |
|---|---|---|---|---|
| Eg | Structure | Name | (min) | m/z (ES⁺) |
| 66 | 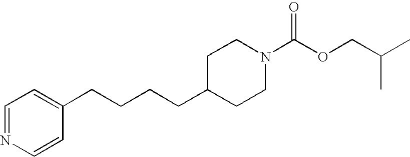 | 4-(4-Pyridin-4-ylbutyl)piperidine-1-carboxylic acid isobutylester | 2.20 | 319.3[M + H]⁺ |
| 67 | 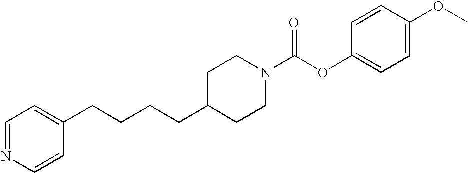 | 4-(4-Pyridin-4-ylbutyl)piperidine-1-carboxylic acid 4-methoxyphenyl ester | 2.22 | 369.3[M + H]⁺ |
| 68 | 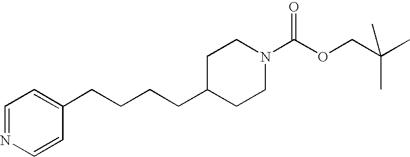 | 4-(4-Pyridin-4-ylbutyl)piperidine-1-carboxylic acid 2,2-dimethylpropyl ester | 2.34 | 333.3[M + H]⁺ |
| 69 | 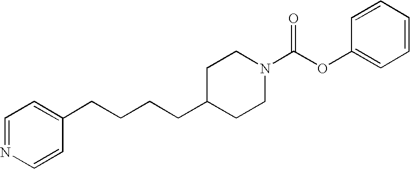 | 4-(4-Pyridin-4-ylbutyl)piperidine-1-carboxylic acid phenylester | 2.38 | 339.4[M + H]⁺ |
| 70 | 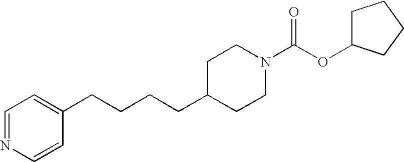 | 4-(4-Pyridin-4-ylbutyl)piperidine-1-carboxylic acidcyclopentyl ester | 2.39 | 331.3 [M +H]⁺ |
| 71 | 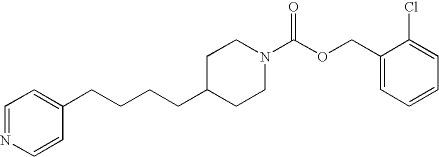 | 4-(4-Pyridin-4-ylbutyl)piperidine-1-carboxylic acid 2-chlorobenzyl ester | 2.50 | 387.3[M + H]⁺ |
| 72 | 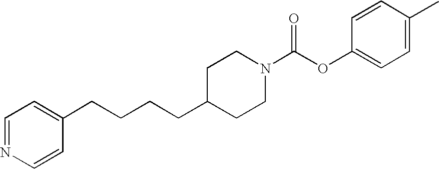 | 4-(4-Pyridin-4-ylbutyl)piperidine-1-carboxylic acid p-tolylester | 2.35 | 353.3[M + H]⁺ |
| 73 | 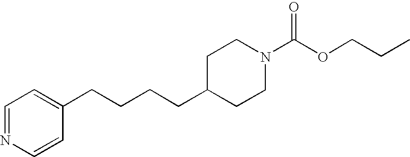 | 4-(4-Pyridin-4-ylbutyl)piperidine-1-carboxylic acid propylester | 2.06 | 305.3[M + H]⁺ |
| 74 | 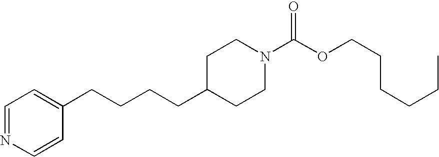 | 4-(4-Pyridin-4-ylbutyl)piperidine-1-carboxylic acid hexylester | 2.55 | 347.4[M + H]⁺ |
| 75 | 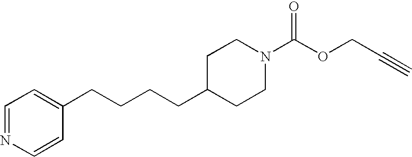 | 4-(4-Pyridin-4-ylbutyl)piperidine-1-carboxylic acid prop-2-ynyl ester | 2.09 | 301.3[M + H]⁺ |
| 76 | 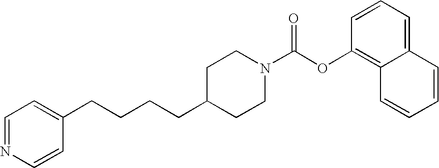 | 4-(4-Pyridin-4-ylbutyl)piperidine-1-carboxylic acidnaphthalen-1-yl ester | 2.58 | 389.3[M + H]⁺ |
| 77 | 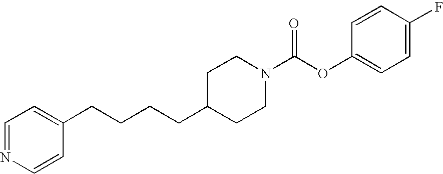 | 4-(4-Pyridin-4-ylbutyl)piperidine-1-carboxylic acid 4-fluorophenyl ester | 2.34 | 357.3[M + H]⁺ |
| 78 | 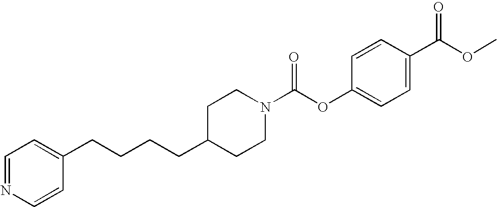 | 4-(4-Pyridin-4-ylbutyl)piperidine-1-carboxylic acid 4-methoxycarobnylphenylester | 2.35 | 397.3[M + H]⁺ |
| 79 | 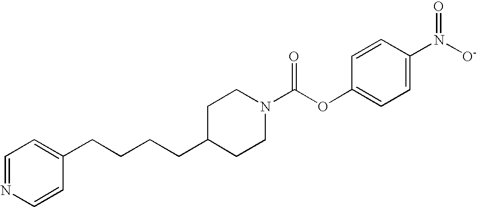 | 4-(4-Pyridin-4-ylbutyl)piperidine-1-carboxylic acid 4-nitrophenyl ester | 3.22 | 384.4[M + H]⁺ |
| 80 | 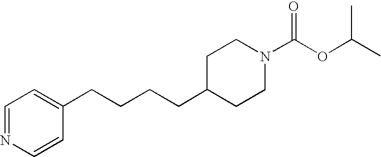 | 4-(4-Pyridin-4-ylbutyl)piperidine-1-carboxylic acid isopropylester | 2.86 | 305.1[M + H]⁺ |
| 81 | 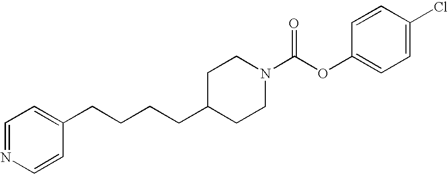 | 4-(4-Pyridin-4-ylbutyl)piperidine-1-carboxylic acid 4-chlorophenyl ester | 3.19 | 373.3[M + H] |
| 82 | 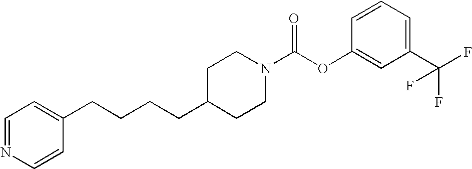 | 4-(4-Pyridin-4-ylbutyl)piperidine-1-carboxylic acid 3-trifluoromethylphenylester | 3.32 | 407.5[M + H]⁺ |
| 83 | 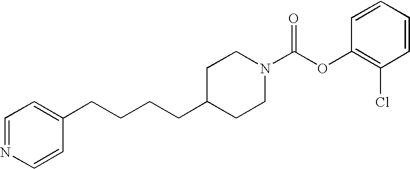 | 4-(4-Pyridin-4-ylbutyl)piperidine-1-carboxylic acid 2-chlorophenyl ester | 3.22 | 373.3[M + H]⁺ |
| 84 | 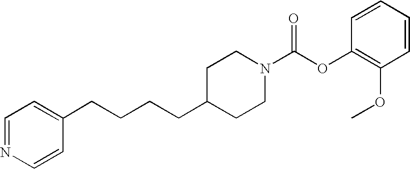 | 4-(4-Pyridin-4-ylbutyl)piperidine-1-carboxylic acid 2-methoxyphenyl ester | 3.04 | 369.3[M + H]⁺ |
| 85 | 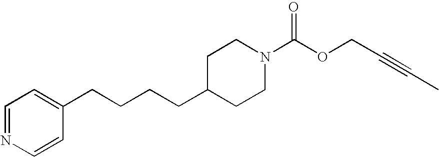 | 4-(4-Pyridin4-yl-butyl)piperidine-1-carboxylic acid but-2-ynyl ester | 2.81 | 315.2[M + H]⁺ |
| 86 | 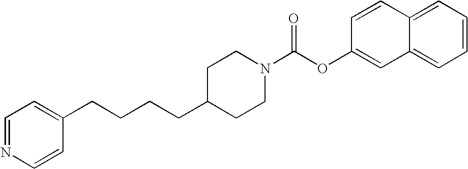 | 4-(4-Pyridin-4-ylbutyl)piperidine-1-carboxylic acidnaphthalen-2-ylester | 3.37 | 389.3[M + H]⁺ |
| 87 | 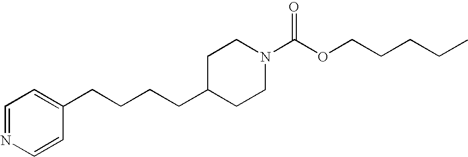 | 4-(4-Pyridin-4-ylbutyl)piperidine-1-carboxylic acid pentylester | 3.27 | 333.3[M + H]⁺ |

### Example 88

### 4-(4-Pyridin-4-ylbutyl)piperidine-1-carboxylic acid o-tolyl ester

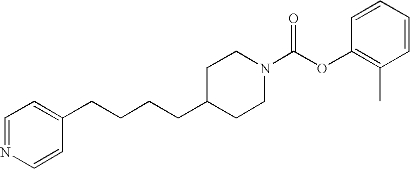

A solution of triphosgene (1.5 mL of a 0.0407 M solution in THF, 61 μmol) was added to 2-methylphenol (19.8 mg, 183 μmol) under argon and NEt₃ (51 μL, 366 μmol) in anhydrous THF (0.5 mL) was then introduced. After stirring for 0.5 h, precisely 1.0 mL of the resulting mixture was added to a stirred solution of 4-(4-piperidin-4-ylbutyl)pyridine (Preparation 5, 20 mg, 91.5 μmol) in anhydrous THF (1.0 mL). After 0.5 h, the reaction was diluted with EtOAc (12 mL), washed with water (4 mL) and brine (4 mL), then dried (MgSO₄). The solvent was evaporated and the residue purified by column chromatography (EtOAc) to afford the title compound: RT=3.14 min; m/z (ES⁺)=353.2 [M+H]⁺.

The compounds listed in Table 9 were synthesised according to procedures similar to that described in Example 88.

TABLE 9

| | | | RT | m/z |
|---|---|---|---|---|
| Eg | Structure | Name | (min) | (ES⁺) |
| 89 | 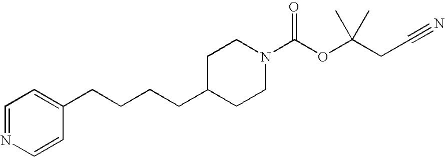 | 4-(4-Pyridin-4-ylbutyl)piperidine-1-carboxylic acid 2-cyano-1,1-dimethylethyl ester | 2.90 | 344.3[M + H]⁺ |
| 90 | 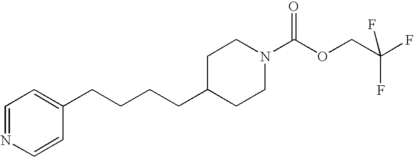 | 4-(4-Pyridin-4-ylbutyl)piperidine-1-carboxylic acid 2,2,2-trifluoroethyl ester | 2.90 | 345.1[M + H]⁺ |
| 91 | 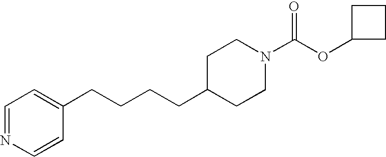 | 4-(4-Pyridin-4-ylbutyl)piperidine-1-carboxylic acid cyclobutylester | 2.86 | 317.2[M + H]⁺ |
| 92 | 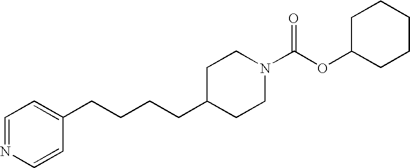 | 4-(4-Pyridin-4-ylbutyl)piperidine-1-carboxylic acid cyclohexylester | 3.15 | 345.2[M + H]⁺ |
| 93 | 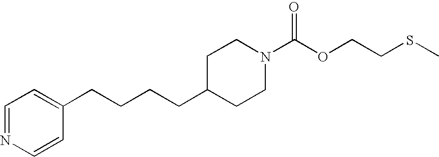 | 4-(4-Pyridin-4-ylbutyl)piperidine-1-carboxylic acid 2-methylsulfanylethyl ester | 2.76 | 337.1[M + H]⁺ |
| 94 | 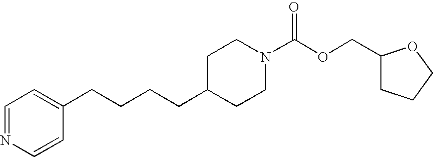 | 4-(4-Pyridin-4-ylbutyl)piperidine-1-carboxylic acidtetrahydrofuran-2-ylmethylester | 2.64 | 347.2[M + H]⁺ |

### Example 95

### 2-[4-(4-Pyridin-4-ylbutyl)piperidin-1-yl]propionic acid ethyl ester

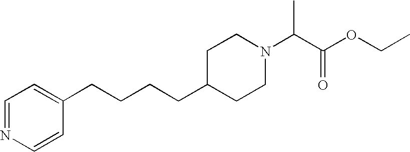

A solution of ethyl-2-bromopropionate in anhydrous CH₂Cl₂ (1 mL) was added to a stirred solution of 4-(4-piperidin-4-ylbutyl)pyridine (Preparation 5, 20 mg, 91.5 μmol) and NEt₃ (19 μL, 140 μmol) in anhydrous CH₂Cl₂ (1.0 mL). After 18 h, the solvent was evaporated and the residue purified by column chromatography (EtOAc) to afford the title compound: RT=1.77 min; m/z (ES⁺) 319.2 [M+H]⁺.

The compounds listed in Table 10 were produced using protocols similar to that described in Example 95.

TABLE 10

| | | | RT | m/z |
|---|---|---|---|---|
| Eg | Structure | Name | (min) | (ES⁺) |
| 96 | 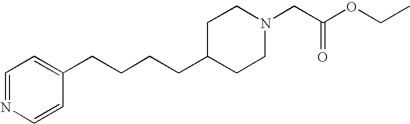 | [4-(4-Pyridin-4-ylbutyl)piperidin-1-yl]acetic acid ethyl ester | 1.67 | 305.2[M + H]⁺ |
| 97 | 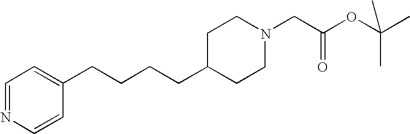 | [4-(4-Pyridin-4-ylbutyl)piperidin-1-yl]acetic acid tert-butylester | 1.86 | 333.2[M + H]⁺ |

### Example 98

### Oxo-[4-(4-pyridin-4-ylbutyl)piperidin-1-yl]acetic acid methyl ester

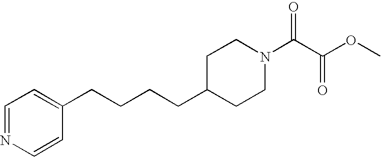

A stirred solution of 4-(4-piperidin-4-ylbutyl)pyridine (Preparation 5, 70 mg, 320 μmol) and NEt₃ (89 μL, 640 μmol) in anhydrous CH₂Cl₂ (3.0 mL) was treated with neat methyl chlorooxoacetate (31 μL, 335 μmol). After 5 min the solvent was evaporated and the residue purified by column chromatography (EtOAc) to afford the title compound: RT=2.24 min; m/z (ES⁺)=305.1 [M+H]⁺.

### Example 99

### 2-[4-(4-Pyridin-4-ylbutyl)piperidin-1-yl]pyrimidine

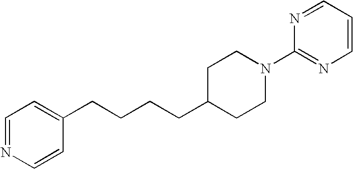

A solution of 4-(4-piperidin-4-ylbutyl)pyridine (Preparation 5, 20 mg, 91.5 μmol), 2-bromopyrimidine (17.5 mg, 110 μmol) and 1,8-diazabicyclo[5.4.0]undec-7-ene (30 μL, 200 μmol) in 1,4-dioxane (0.4 mL) was stirred at RT for 52 h. The solvent was removed and the residue purified by column chromatography (EtOAc) to afford the title compound: RT=2.31 min; m/z (ES⁺)=297.1 [M+H]⁺.

### Example 100

### 4-(4-Pyridin-4-ylbutyl)-3,4,5,6-tetrahydro-2H-[1,2′]bipyridinyl

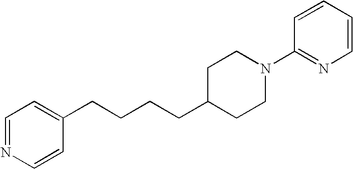

Neat 1,8-diazabicyclo[5.4.0]undec-7-ene (21.5 μL, 140 μmol) was added to a solution of 4-(4-piperidin-4-ylbutyl)pyridine (Preparation 5.21 mg, 96 μmol) in 2-fluoropyridine (0.4 mL) and the stirred mixture heated at 80° C. After 6 h the solvent was removed and the residue purified by column chromatography (EtOAc) to afford the title compound: RT=1.99 min; m/z (ES⁺)=296.1 [M+H]⁺.

### Example 101

### 4-(2,4-Dioxo-4-pyridin-4-ylbutyl)piperidine-1-carboxylic acid tert-butyl ester

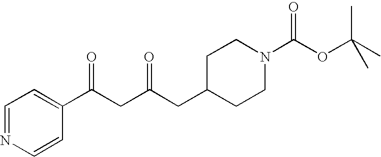

Lithium diisopropylamide (2.16 mL of a 2 M solution in TIF, 4.32 mmol) was added to a flask charged with anhydrous THF (15.3 mL). After cooling to −78° C., neat 4-acetylpyridine (0.48 mL, 4.32 mmol) was introduced in a dropwise manner and the resulting solution stirred at −78° C. for 45 min. To this mixture was then added slowly via cannula solution prepared by adding 1,1′-carbonyldiimidazole (0.666 g, 4.11 mmol) in one portion to a solution of 4-carboxymethylpiperidine-1-carboxylic acid tert-butyl ester (1 g, 4.11 mmol) in anhydrous THF (7.6 mL) and stirring at room temperature for 45 min. The resulting reaction mixture was slowly brought to RT and stirred for a further 2 h. then diluted with EtOAc (150 mL). The organic phase was washed with 10% aqueous citric acid (2×15 mL), saturated aqueous NaHCO₃ (2×15 mL) and brine (15 mL) then dried (MgSO₄). The solvent was removed and the residue purified by RP-HPLC to afford the title compound: RT=3.72 min; m/z (ES⁺)=347.2 [M+H]⁺.

### Example 102

### 4-(3,5-Dioxo-5-pyridin-4-yl-pentyl)piperidine-1-carboxylic acid tert-butyl ester

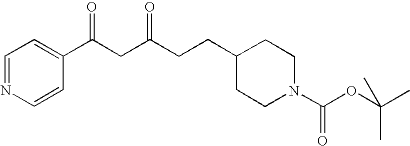

4-Acetylpyridine with 4-(2-carboxyethyl)piperidine-1-carboxylic acid tert-butyl ester in a manner analogous to that described in Example 101 to give the title compound: RT=3.87 min; m/z (ES⁺)=361.3 [M+H]⁺.

### Example 103

### 4-[1-(2-Cyanopyridin-4-yl)vinyloxycarbonylmethyl]piperidine-1-carboxylic acid tert-butyl ester

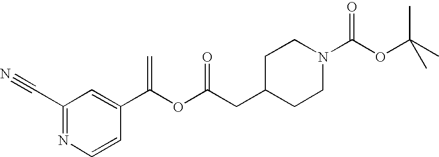

A stirred solution of diisopropylamine (140 μL, 1.03 mmol) in anhydrous THF (2.5 mL) was cooled to −10° C. and n-butyllithium (410 μL of a 2.5 M solution in hexane, 1.03 mmol) added. After 20 min, the solution was cooled to −78° C. and 4-acetylpyridine-2-carbonitrile (150 mg, 1.03 mmol) in anhydrous TIF (2.5 mL) was introduced and stirring continued for 45 min. To this was then added a mixture prepared by treating a solution of 4-carboxymethylpiperidine-1-carboxylic acid tert-butyl ester (275 mg, 1.13 mmol) and NEt₃ (160 μL, 1.13 mmol) in dry THF (5 mL) at 0° C. with isobutylchloroformate (147 μL, 1.13 mmol) and then stirring at room temperature for 1 h. The resulting reaction was allowed to warm to room temperature and, after 18 h, the solvent was evaporated and the residue dissolved in EtOAc (20 mL). The solution was washed with saturated aqueous NaHCO₃ (5 mL) and brine (5 mL) then dried (MgSO₄) and evaporated. Column chromatography (IH-EtOAc, 7:3) afforded the required compound: RT=3.84 min; m/z (ES⁺)=372.07 [M+H]⁺.

### Example 104

### 4-(2-Hydroxy-4-pyridin-4-ylbutyl)piperidine-1-carboxylic acid tert-butyl ester

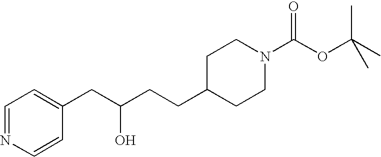

Diisopropylamine (4.1 mL, 29.26 mmol) in anhydrous THF (30 mL) was cooled to −10° C. and n-butyllithium (11.25 mL of a 2.5 M solution in hexane, 28.13 mmol) added dropwise. After stirring for 15 min, the solution was cooled to −78° C. and 4-methylpyridine (2.74 mL, 28.13 mmol) in anhydrous THF (25 mL) was introduced slowly over 45 min, ensuring the temperature did not exceed −60° C. during the addition. After again cooling to −78° C., a solution of 4-(3-oxopropyl)piperidine-1-carboxylic acid tert-butyl ester (4.55 g, 18.85 mmol) in anhydrous THF (25 mL) was added slowly over 25 min then stirred for a further 2 h before being allowed to warm to room temperature. The reaction was quenched with saturated aqueous NH₄Cl (20 mL) and diluted with EtOAc (300 mL). The organic phase was separated, washed with brine (30 mL), dried (MgSO₄) then evaporated to dryness. The solvent was removed and the residue purified by column chromatography (IH-EtOAc, 1:4) to furnish the title compound: RT=1.85 min; m/z (ES⁺)=335.4 [M+H]⁺.

### Example 105

### 4-(2-Hydroxy-4-pyridin-4-ylbutyl)piperidine-1-carboxylic acid tert-butyl ester

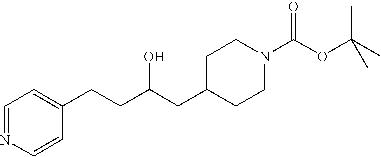

4-Methylpyridine was treated with lithiumdiisopropylmide and then reacted with 4-oxiranylmethylpiperidine-1-carboxylic acid tert-butyl ester using a procedure analogous to that described in Example 104 to afford the title compound: RT=2.57 min; m/z (ES⁺)=335.3 [M+H]⁺.

### Example 106

### 4-(2-Hydroxy-4-oxo-4-pyridin-4-ylbutyl)piperidine-1-carboxylic acid tert-butyl ester

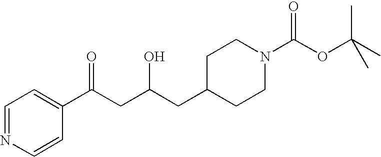

Diisopropylamine (150 μL, 1.46 mmol) in anhydrous THF (2 mL) was cooled to −10° C. and n-butyllithium (560 μL of a 2.5 M solution in hexane, 1.40 mmol) was added. After stirring for 15 min, the solution was cooled to −78° C. and 1-pyridin-4-ylethanone (154 μL, 1.40 mmol) introduced. After 30 min, a solution of 4-(2-oxoethyl)piperidine-1-carboxylic acid tert-butyl ester (254 mg, 1.12 mmol) in anhydrous THF (1.5 mL) was added and the reaction stirred for a further 2 h before being quenched with saturated aqueous NaHCO₃ (2 mL). Following dilution with EtOAc (50 mL), the organic phase was separated and dried (MgSO₄) then evaporated. The residue was purified by column chromatography (EtOAc) to give the title compound: RT=3.15 min; m/z (ES⁺)=349.2 [M+H]⁺.

### Example 107

### 4-[4-(2-Cyanopyridin-4-yl)-2-hydroxy-4-oxobutyl]piperidine-1-carboxylic acid tert-butyl ester

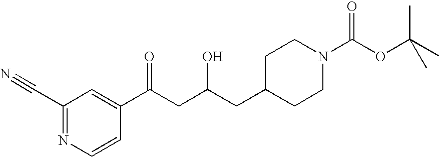

4-Acetylpyridine-2-carbonitrile was reacted with 4-(2-oxoethyl)piperidine-1-carboxylic acid tert-butyl ester, using the procedure described in Example 106 to afford the title compound: RT=3.45 min; m/z (ES⁺)=374.1 [M+H]⁺.

### Example 108

### 4-(1-Hydroxy-4-pyridin-4-ylbutyl)piperidine-1-carboxylic acid tert-butyl ester

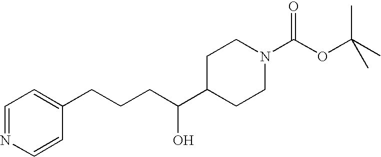

A suspension of 4-(3-bromopropyl)pyridine hydrobromide (297 mg, 1.057 mmol) in Et₂O 20 mL) was washed with saturated aqueous Na₂CO₃ (5 mL) and the ethereal solution dried (MgSO₄) before being concentrated to about 2 mL in volume. Following dilution with hexane (8 mL) the stirred solution was cooled to −78° C. and treated with t-butyllithium (1.33 mL of a 1.5 M solution in pentane, 2.0 mmol). After 30 min, a solution of 4-formylpiperidine-1-carboxylic acid tert-butyl ester in dry Et₂O (2.0 mL) was added and stirring continued for 1 h. The reaction was then quenched with saturated aqueous NH₄Cl (2 mL) and diluted with EtOAc (40 mL). After washing the organic phase with brine (5 mL) and drying (MgSO₄), the solvent was removed and the residue purified by column chromatography (EtOAc-MeOH, 25:1) to give the title compound: RT=1.83 min; m/z (ES⁺)=335.3 [M+H]⁺.

### Example 109

### (Z)-4-(4-Oxo-4-pyridin-4-ylbut-2-enyl)piperidine-1-carboxylic acid tert-butyl ester

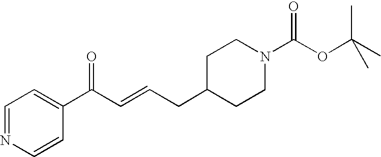

A solution of 4-(2-hydroxy-4-oxo-4-pyridin-4-ylbutyl)piperidine-1-carboxylic acid tert-butyl ester (Example 106, 124 mg, 356 μmol) and NEt₃ (150 μL, 1.07 mmol) was cooled to 0° C. and methanesulfonyl chloride (30.5 μL, 400 μmol) added. After stirring at room temperature for 4 h, the reaction was diluted with CH₂Cl₂ (20 mL) and washed with brine (4 mL) and dried (MgSO₄). Evaporation of the solvent and purification of the residue by column chromatography (IH-EtOAc, 1:4) gave the title compound: RT=3.56 min; m/z (ES⁺)=331.3 [M+H]⁺.

### Examples 110 and 111

### 4-(4-Oxo-4-pyridin-4-ylbutyl)piperidine-1-carboxylic acid tert-butyl ester and 4-(4-hydroxy-4-pyridin-4-ylbutyl)piperidine-1-carboxylic acid tert-butyl ester

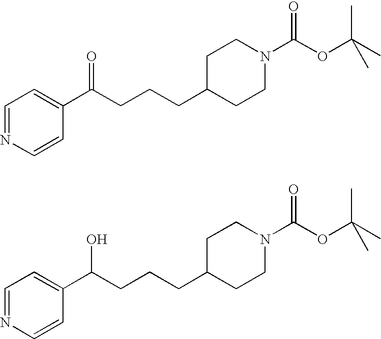

A slurry of Pd (80 mg, 10% on C, 75 μmol) in EtOAc (1 mL) was added to a stirred solution of (Z)-4-(4-oxo-4-pyridin-4-ylbut-2-enyl)piperidine-1-carboxylic acid tert-butyl ester (Example 109, 457 mg, 1.385 mmol) in EtOAc (15 mL) and a hydrogen atmosphere introduced. After 2.5 h, the mixture was filtered through a pad of Celite, washing with a little EtOAc, and the solvent evaporated. Purification by column chromatography (IH-EtOAc, 3:7) afforded firstly the title ketone: RT=3.74 min; m/z (ES⁺)=333.3 [M+H]⁺, and subsequently the title alcohol: RT=2.67 min; m/z (ES⁺)=335.3 [M+H]⁺.

### Example 112

### 4-[4-(2-Cyanopyridin-4-yl)-2-hydroxybutyl]piperidine-1-carboxylic acid tert-butyl ester

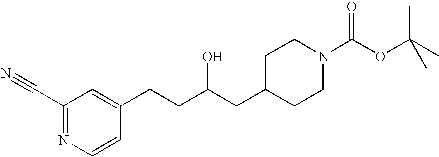

Tetra-n-butylammonium fluoride (8.2 ml of a 1 M solution in THF, 8.2 mmol was added to a stirred solution of 4-[4-(2-cyanopyridin-4-yl)-2-trimethylsilanyloxybutyl]piperidine-1-carboxylic acid tert-butyl ester (Preparation 8, 3.212 g, 7.452 mmol) in THF (25 mL). After 1 h the reaction was poured into Et₂O (250 mL) and washed with water (25 mL), brine (25 mL) then dried (MgSO₄). The solvent was removed and the residue purified by column chromatography (IH-EtOAc, 3:7) to afford the title compound: RT=3.47 min; m/z (ES⁺)=360.2 [M+H]⁺.

The alcohols listed in Table 11 were produced from the corresponding silyl ethers in a manner similar to that described in Example 112.

TABLE 11

| | | | RT | m/z |
|---|---|---|---|---|
| Eg | Structure | Name | (min) | (ES⁺) |
| 113 | 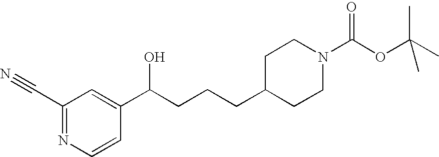 | 4-[4-(2-Cyanopyridin-4-yl)-4-hydroxybutyl]piperidine-1-carboxylic acid tert-butylester | 3.72 | 360.3[M + H]⁺ |
| 114 | 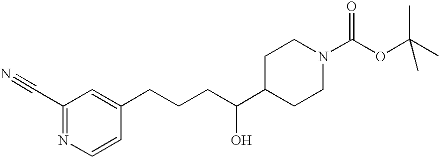 | 4-[4-(2-Cyanopyridin-4-yl)-1-hydroxybutyl]piperidine-1-carboxylic acid tert-butylester | 3.49 | 360.2[M + H]⁺ |

### Example 115

### 4-(2-Oxo-4-pyridin-4-ylbutyl)piperidine-1-carboxylic acid tert-butyl ester

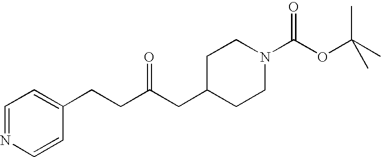

Jones reagent (1.7 mL) was added at to stirred solution of 4-(2-hydroxy-4-pyridin-4-ylbutyl)piperidine-1-carboxylic acid tert-butyl ester (Example 105, 167 mg, 499 μmol) in acetone (4 mL) at 0° C. The reaction mixture was stirred at this temperature for 4.5 h then basified to pH 9 by the careful addition of saturated aqueous Na₂CO₃. After dilution with water (2 mL) and EtOAc (20 mL), the precipitated solids were removed by filtration through a pad of Celite. The organic phase was then separated and washed with brine (5 mL), dried (MgSO₄) and evaporated to give the title compound: RT=2.56 min; m/z (ES⁺)=333.3 [M+H]⁺.

The compounds in Table 12 were synthesised from the corresponding alcohols using procedures similar to that described in Example 115.

TABLE 12

| | | | RT | m/z |
|---|---|---|---|---|
| Eg | Structure | Name | (min) | (ES⁺) |
| 116 | 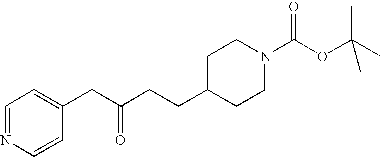 | 4-(3-Oxo-4-pyridin-4-ylbutyl)piperidine-1-carboxylic acid tert-butylester | 2.19 | 333.4[M + H]⁺ |
| 117 | 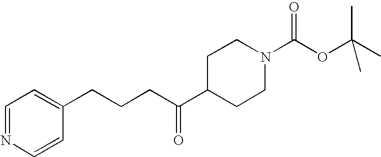 | 4-(4-Pyridin-4-yl-butyryl)piperidine-1-carboxylic acid tert-butylester | 2.56 | 333.4[M + H]⁺ |
| 118 | 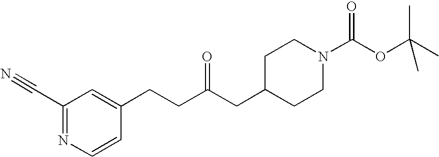 | 4-[4-(2-Cyanopyridin-4-yl)-2-oxobutyl]piperidine-1-carboxylic acid tert-butyl ester | 3.65 | 358.2[M + H]⁺ |
| 119 | 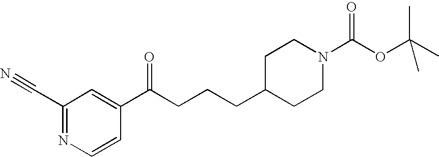 | 4-[4-(2-Cyanopyridin-4-yl)butyryl]piperidine-1-carboxylic acid tert-butylester | 3.95 | 358.2[M + H]⁺ |

### Example 120

### 4-[4-(2-Cyanopyridin-4-yl)butyryl]piperidine-1-carboxylic acid tert-butyl ester

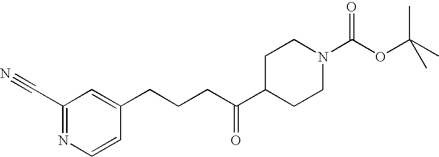

Using a procedure similar to that described in Example 59, 4-(4-pyridin-4-yl-butyryl)piperidine-1-carboxylic acid tert-butyl ester was converted to the title compound: RT=3.74 min; m/z (ES⁺)=358.2 [M+H]⁺.

### Example 121

### 4-(3-Methylamino-4-pyridin-4-ylbutyl)piperidine-1-carboxylic acid tert-butyl ester

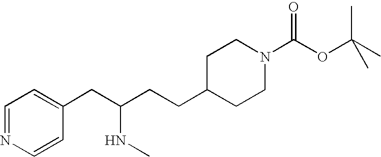

Ti(OⁱPr)₄ (107 μL, 362 μmol) was added to a stirred solution of NEt₃ (51 μL, 362 μmol), 4-(3-oxo-4-pyridin-4-ylbutyl)piperidine-1-carboxylic acid tert-butyl ester (Example 116, 60 mg, 181 μmol) and methylamine hydrochloride (24.4 mg, 362 μmol) in EtOH (1 mL). After 3 h, NaBH₄ (11 mg, 290 μmol) was added and stirring continued for a further 3 h. The reaction mixture was diluted with CH₂Cl₂ (20 mL) and washed with water (4 mL) and brine (4 mL) then dried (MgSO₄). Following removal of the solvent, the crude product was purified by column chromatography (MeOH-EtOAc, 1:9 then MeOH-EtOAc-NEt₃, 1:0.5:8.5) to give the title compound: RT=2.31 min; m/z (ES⁺)=348.3 [M+H]⁺.

The compounds listed in Table 13 were synthesised from the corresponding ketones using procedures similar to that described in Example 121.

TABLE 13

| | | | RT | m/z |
|---|---|---|---|---|
| Eg | Structure | Name | (min) | (ES⁺) |
| 122 | 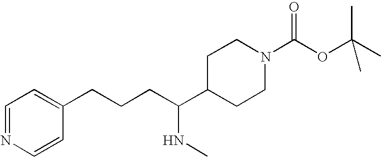 | 4-(1-Methylamino-4-pyridin-4-ylbutyl)piperidine-1-carboxylic acid tert-butylester | 2.59 | 373.2[M + H]⁺ |
| 123 | 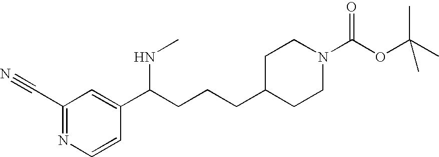 | 4-[4-(2-Cyanopyridin-4-yl)-4-methylaminobutyl]piperidine-1-carboxylicacid tert-butyl ester | 2.79 | 373.2[M + H]⁺ |
| 124 | 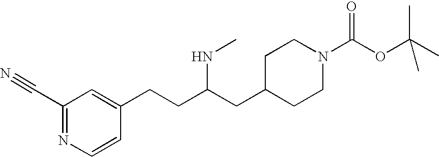 | 4-[4-(2-Cyano-pyridin-4-yl)-2-methylamino-butyl]piperidine-1-carboxylic acid tert-butylester | 2.59 | 373.2[M + H]⁺ |

### Example 125

### 4-(1-Dimethylamino-4-pyridin-4-ylbutyl)piperidine-1-carboxylic acid tert-butyl ester

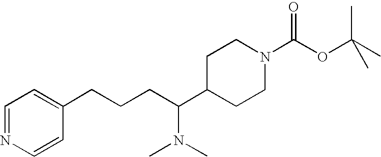

A stirred solution of 4-(1-methylamino-4-pyridin-4-ylbutyl)piperidine-1-carboxylic acid tert-butyl ester (Example 122, 22 mg, 60 μmol), formaldehyde (8 mL of a 37% aqueous solution, 900 μmol) in CH₂Cl₂ (5 mL) was treated with sodium triacetoxyborohydride (20 mg, 900 μmol). After 18 h, the reaction mixture was washed with saturated aqueous NaHCO₃ (2 mL) and dried (MgSO₄). Evaporation of the solvent afforded the title compound: RT=2.52 min; m/z (ES⁺)=362.14 [M+H]⁺.

The compounds listed in Table 14 were synthesised from the corresponding amines using procedures similar to that described in Example 125.

TABLE 14

| | | | RT | m/z |
|---|---|---|---|---|
| Eg | Structure | Name | (min) | (ES⁺) |
| 126 | 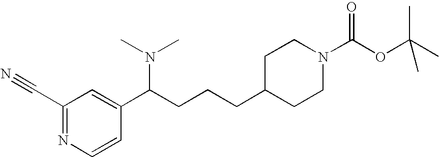 | 4-[4-(2-Cyanopyridin-4-yl)-4-dimethylaminobutyl]-piperidine-1-carboxylicacid tert-butyl ester | | |
| 127 | 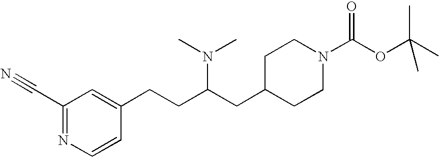 | 4-[4-(2-Cyanopyridin-4-yl)-2-dimethylaminobutyl]-piperidine-1-carboxylicacid tert-butyl ester | 2.62 | 387.2[M + H]⁺ |

### Example 128

### 4-[2-(2-Carbamoylpyridin-4-ylmethoxy)ethyl]piperidine-1-carboxylic acid tert-butyl ester

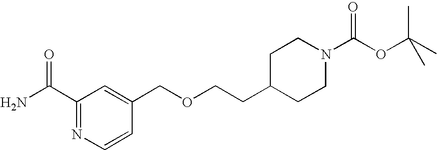

A stirred solution of 4-[2-(2-cyanopyridin-4-ylmethoxy)ethyl]piperidine-1-carboxylic acid tert-butyl ester (Example 62, 50 mg, 145 μmol) in anhydrous TIF (3 mL) was treated with methylmagnesium chloride (0.1 mL of a 3 M solution in THF, 300 μmol). After 18 h, the reaction mixture was poured into EtOAc (20 mL) and washed with saturated aqueous NH₄Cl (3 mL) and brine (5 mL). The solvent was removed and column chromatography afforded the title compound: RT=3.29 min; m/z (ES⁺)=364.1 [M+H]⁺.

### Example 129

### 4-[2-(2-Ethynylpyridin-4-ylmethoxy)ethyl]piperidine-1-carboxylic acid tert-butyl ester

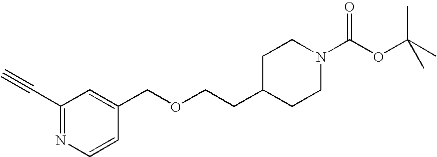

An argon-purged solution of 4-[2-(2-bromopyridin-4-ylmethoxy)ethyl]piperidine-1-carboxylic acid tert-butyl ester (Example 39, 75 mg, 190 μmol) in anhydrous DMF (3 mL) was treated sequentially with NEt₃ (30 μL, 215 μmol), trimethylsilylacetylene (50 μmL, 355 μmol), copper(I) iodide (2 mg, 10 μmol) and dichlorobis(triphenylphosphine)palladium(II). After stirring 17 h, the same quantities of NEt₃, trimethylsilylacetylene, copper(I) iodide and dichlorobis(triphenylphosphine)palladium(II) were again added. After 44 h the solvent was removed and the residue purified by column chromatography (IH-EtOAc, 1:1) to afford 4-[2-(2-trimethylsilanylethynylpyridin-4-ylmethoxy)ethyl]piperidine-1-carboxylic acid tert-butyl ester: RT=4.55 min; m/z (ES⁺)=417.1 [M+H]⁺. A mixture of this acetylene in methanol (10 mL) and K₂CO₃ (45 mg, 325 μmol) was stirred for 16 h, the solvent evaporated and the residue purified by column chromatography (IH-EtOAc, 1:1) to give the title compound: RT=3.74 min; m/z (ES⁺)=345.1 [M+H]⁺.

### Examples 133 and 134

### 4-[(E)-4-(2-Methylpyridin-4-yl)but-3-enyl]piperidine-1-carboxylic acid tert-butyl ester and 4-[(Z)-4-(2-Methylpyridin-4-yl)but-3-enyl]piperidine-1-carboxylic acid tert-butyl ester

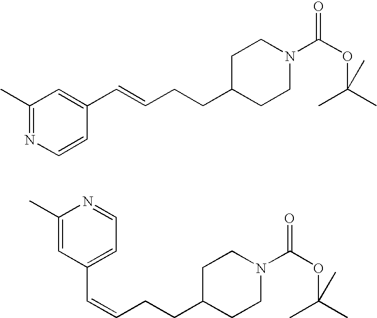

(2-Methylpyridin-4-ylmethyl)triphenylphosphonium chloride (Preparation 10) was reacted with 4-(3-oxopropyl)piperidine-1-carboxylic acid tert-butyl ester, using a method similar to that described in Examples 52 and 53, to afford the title (E)-olefin: δ₆H (CDCl₃) 1.12 (dq, 2H), 1.44 (t, 2H), 1.47 (s, 9H), 1.61 (s, 1H), 1.69 (d, 2H), 2.27 (q, 2H), 2.54 (s, 3H), 2.69 (t, 2H), 4.09 (m, 2H), 6.30 (d, 1H), 6.43 (dt, 1H), 7.02 (d, 1H), 7.07 (s, 1H), 8.39 (d, 1H); and the title (Z)-olefin: δ₆H (CDCl₃) 1.09 (m, 2H), 1.41 (t, 2H), 1.46 (s, 9H), 1.58 (s, 1H), 1.62 (d, 2H), 2.34 (q, 2H), 2.56 (s, 3H), 2.66 (t, 2H), 4.06 (m, 2H), 5.80 (dt, 1H), 6.32 (d, 1H), 6.96 (d, 1H), 7.00 (s, 1H), 8.45 (d, 1H).

### Example 135

### 4-[4-(2-Methylpyridin-4-yl)butyl]piperidine-1-carboxylic acid tert-butyl ester

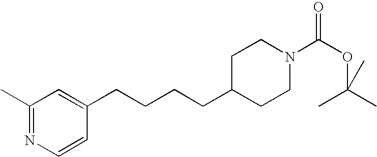

A mixture of (E)- and (Z)-4-[4-(2-methylpyridin-4-yl)but-3-enyl]piperidine-1-carboxylic acid tert-butyl esters (Examples 133 and 134) was hydrogenated over a Pd catalyst, using a similar procedure to that described in Example 56, to afford the title compound: RT=2.70 min; m/z (ES⁺)=333.2 [M+H]⁺.

### Example 136

### 4-Hydroxy-4-[4-(2-methylpyridin-4-yl)butyl]piperidine-1-carboxylic acid tert-butyl ester

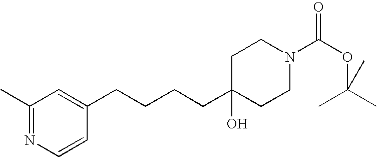

(2-Methylpyridin-4-ylmethyl)triphenylphosphonium chloride was reacted with 2-hydroxy-1-oxa-8-azaspiro[4.5]decane-8-carboxylic acid tert-butyl ester (Preparation 11) using a method similar to that described for Examples 52 and 53 to afford a mixture of (E)- and (Z)-4-hydroxy-4-[4-(2-methylpyridin-4-yl)but-3-enyl]piperidine-1-carboxylic acid tert-butyl ester: RT=2.37 min; m/z (ES+)=347.3 [M+H]⁺. A sample of this mixture of olefins was dissolved in EtOH and hydrogenated over a Pd catalyst, using a similar procedure to that described in Example 56, to give the title compound: RT=2.32 min; m/z (ES+)=349.3 [M+H]⁺.

The biological activity of representative compounds of the invention was tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast alpha-cell GPCR, Ste3 has been deleted to allow for a homologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing P-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR116 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR116 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells are added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein, allowing a fluorimetric read-out. 20 μl per well of 500 μM FDG/2.5% Triton X100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR116 was established and this cell line was used to investigate the effect of compounds of the invention on intracellular levels of cyclic AMP (cAMP). The cells monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol. Compounds of the invention showed a concentration-dependant increase in intracellular cAMP level.

Compounds of the invention showed a concentration-dependant increase in intracellular cAMP level and generally had an EC₅₀ of <10 μM. Compounds showing an EC₅₀ of less than 1 um in the cAMP assay may be preferred.

### IN Vivo Feeding Study

The effect of compounds of the invention on body weight and food and water intake may be examined in freely-feeding male Sprague-Dawley rats maintained on reverse-phase lighting. Test compounds and reference compounds are dosed by appropriate routes of administration (e.g. intraperitoneally or orally) and measurements made over the following 24 h. Rats are individually housed in polypropylene cages with metal grid floors at a temperature of 21±4° C. and 55±20% humidity. Polypropylene trays with cage pads are placed beneath each cage to detect any food spillage. Animals are maintained on a reverse phase light-dark cycle (lights off for 8 h from 09.30-17.30 h) during which time the room was illuminated by red light. Animals have free access to a standard powdered rat diet and tap water during a two week acclimatization period. The diet is contained in glass feeding jars with aluminum lids. Each lid has a 3-4 cm hole in it to allow access to the food. Animals, feeding jars and water bottles are weighed (to the nearest 0.1 g) at the onset of the dark period. The feeding jars and water bottles are subsequently measured 1, 2, 4, 6 and 24 h after animals are dosed with a compound of the invention and any significant differences between the treatment groups at baseline compared to vehicle-treated controls.

Selected compounds of the invention showed a statistically significant hypophagic effect at one or more time points at a dose of ≦100 mg/kg.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP assay

HIT-T15 cells were plated in standard culture medium in 96-well plates at 100,000 cells/0.1 ml/well and cultured for 24 hr and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 ul lysis buffer (5 mM HEPES, 0.3% Tween-20, 0.1% BSA, pH 7.4) was added per well and the plate was shaken at 900 rpm for 20 min. Particulate matter was removed by centrifugation at 300 rpm for 5 min, then the samples were transferred in duplicate to 384-well plates, and processed following the Perkin Elmer AlphaScreen cAMP assay kit instructions. Briefly 25 μl reactions were set up containing 8 μl sample, 5 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells were plated in standard culture medium in 12-well plates at 106 cells/1 ml/well and cultured for 3 days and the medium was then discarded. Cells were washed ×2 with supplemented Krebs-Ringer buffer (KRB) containing 119 mM NaCl, 4.74 mM KCl, 2.54 mM CaC₂, 1.19 mM MgSO₄, 1.19 mM KH2PO4, 25 mM NaHCO₃, 10 mM HEPES at pH 7.4 and 0.1% bovine serum albumin. Cells were incubated with 1 ml KRB at 37° C. for 30 min which was then discarded. This was followed by a second incubation with KRB for 30 min, which was collected and used to measure basal insulin secretion levels for each well. Compound dilutions (0, 0.1, 0.3, 1, 3, 10 uM) were then added to duplicate wells in 1 ml KRB, supplemented with 5.6 mM glucose. After 30 min incubation at 37° C. samples were removed for determination of insulin levels. Measurement of insulin was done using the Mercodia Rat insulin ELISA kit, following the manufacturers instructions, with a standard curve of known insulin concentrations. For each well insulin levels were subtracted by the basal secretion level from the pre-incubation in the absence of glucose. Data was analysed using XLfit 3 software.

Representative compounds of the invention were found to increase insulin secretion at an EC₅₀ of less than 10 μM. Compounds showing an EC₅₀ of less than 1 μm in the insulin secretion assay may be preferred.

What is claimed is:

1. A compound of formula (I), or a pharmaceutically acceptable salt thereof: 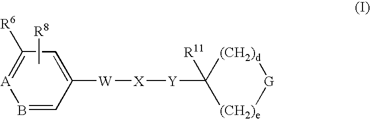
wherein one of A and B is nitrogen and the other is CR¹;
W and Y are independently a bond, an unbranched or a branched C₁₋₃ alkylene or an unbranched or a branched C₂₋₃ alkenylene;
X is selected from CH₂, O, S, CH(OH), CH(halogen), C(O), C(O)O, C(O)S, SC(O), C(O)CH₂S, C(O)CH₂C(OH), C(O)CH₂C(O), OC(O), NR⁵, CH(NR⁵R⁵⁵), C(O)NR², S(O) and S(O)₂;
G is CHR³, N—C(O)OR⁴, N—C(O)NR⁴R⁵, N—C₁₋₄alkylene-C(O)OR⁴, N—C(O)C(O)OR⁴, N—S(O)₂R⁴, N—C(O)R⁴ or N—P(O)(O-Ph)₂; or N-heterocyclyl or N-heteroaryl, either of which may optionally be substituted by one or two groups selected from C₁₋₄alkyl, C₁₋₄alkoxy or halogen;
R¹ is hydrogen, halogen, cyano, C(O)NH₂, C₁₋₄alkyl, SO₂C₁₋₄alkyl, SOC₁₋₄alkyl or SC₁₋₄alkyl;
R² is hydrogen or C₁₋₄ alkyl;
R³ is C₃₋₆ alkyl;
R⁴ is C₁₋₈ alkyl, C₂₋₈ alkenyl or C₂₋₈ alkynyl, any of which may be optionally substituted by one or more halo atoms, NR⁵R⁵⁵, OR⁵, C(O)OR⁵, OC(O)R⁵ or cyano, and may contain a CH₂ group that is replaced by O or S; or a C₃₋₇cycloalkyl, aryl, heterocyclyl, heteroaryl, C₁₋₄alkyleneC₃₋₇cycloalkyl, C₁₋₄alkylenearyl, C₁₋₄alkyleneheterocyclyl or C₁₋₄alkyleneheteroaryl, any of which may be substituted with one or more substituents selected from halo, C₁₋₄ alkyl, C₁₋₄fluoroalkyl, OR⁵, CN, NR⁵R⁵⁵, SO₂Me, NO₂ or C(O)OR⁵;
R⁵ and R⁵⁵ are independently hydrogen or C₁₋₄alkyl; or taken together R⁵ and R⁵⁵ may form a 5 or 6 membered heterocyclic ring;
R⁶ is hydrogen, halogen, CN, C₁₋₄alkyl, C₁₋₄alkoxy, ethynyl, C(O)NR⁷R⁷⁷ or C₁₋₄alkyleneS(O)_{f},
R⁷ and R⁷⁷ are independently hydrogen or C₁₋₄alkyl; or taken together R⁷ and R⁷⁷ may form a 5 or 6 membered heterocyclic ring;
R⁸ is hydrogen, halogen, CN, C₁₋₄alkyl or C₁₋₄alkoxy;
R¹¹ is hydrogen or hydroxy;
d is 0, 1, 2 or 3;
e is 1, 2, 3, 4 or 5;
with the proviso that d+e is 2, 3, 4 or 5; and
f is 0, 1 or 2.

2. A compound according to claim 1, or a pharmaceutically acceptable salt thereof, wherein A is nitrogen.

3. A compound according to either claim 1 or 2, or a pharmaceutically acceptable salt thereof, wherein R¹ is C₁₋₄ alkyl, hydrogen or cyano.

4. A compound according to any one of claims 1 to 3, or a pharmaceutically acceptable salt thereof, wherein R⁶ is hydrogen, methyl or halogen.

5. A compound according to any one of claims 1 to 4, or a pharmaceutically acceptable salt thereof, wherein G is N—C(O)OR⁴, N—C(O)NR⁴R⁵ or N-heteroaryl.

6. A compound according to claim 5, or a pharmaceutically acceptable salt thereof, wherein G is N—C(O)OR⁴.

7. A compound according to any one of claims 1 to 6, or a pharmaceutically acceptable salt thereof, wherein R⁴ represents C₁₋₈ alkyl, C₂₋₈ alkenyl or C₂₋₈ alkynyl optionally substituted by one or more halo atoms or cyano, which may contain a CH₂ group that may be replaced by O or S; or a C₃₋₇cycloalkyl, aryl or C₁₋₄alkyleneC₃₋₇cycloalkyl, any of which may be substituted with one or more substituents selected from halo, C₁₋₄ alkyl, C₁₋₄ fluoroalkyl, OR⁵, CN, NR⁵R⁵⁵, NO₂ or C(O)OC₁₋₄alkyl.

8. A compound according to claim 7, or a pharmaceutically acceptable salt thereof, wherein R⁴ represents C₁₋₈ alkyl, C₂₋₈ alkenyl or C₂₋₈ alkynyl optionally substituted by one or more halo atoms or cyano, which may contain a CH₂ group that may be replaced by O or S; or a C₃₋₇cycloalkyl which may be substituted with one or more substituents selected from halo, C₁₋₄ alkyl, C₁₋₄ fluoroalkyl, OR⁵, CN, NR⁵R⁵⁵, NO₂ or C(O)OC₁₋₄alkyl.

9. A compound according to claim 7 or 8, or a pharmaceutically acceptable salt thereof, wherein the group represented by R⁴ is unsubstituted.

10. A compound according to any one of claims 1 to 9, or a pharmaceutically acceptable salt thereof, wherein d and e each represent 1.

11. A compound according to any one of claims 1 to 9, or a pharmaceutically acceptable salt thereof, wherein d and e each represent 2.

12. A compound according to any one of the preceding claims, or a pharmaceutically acceptable salt thereof, wherein when W is C₂₋₃ alkenylene, the stereochemistry at the double bond is (E).

13. A compound according to any one of the preceding claims, or a pharmaceutically acceptable salt thereof, wherein —W—X—Y— represents a 4 or 5 atom chain.

14. A compound according to claim 1 which is of formula (Ia), or a pharmaceutically acceptable salt thereof: 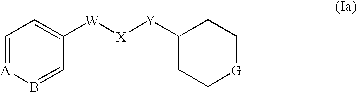
wherein one of A and B is nitrogen and the other is CR¹;
W and Y are independently a bond, C₁₋₃ alkylene or C₂₋₃ alkenylene;
X is selected from CH₂, O, S, CO, CO₂, COS, SCO, COCH₂S, COCH₂CO, OCO, CONR², SO and SO₂;
G is CHR³, NCOOR⁴, or NCONR⁴R⁵;
R¹ is hydrogen, halogen, cyano or C₁₋₄ alkyl;
R² is C₁₋₄alkyl;
R³ is C₃₋₆ alkyl;
R⁴ is C₁₋₆ alkyl, C₂₋₆ alkenyl or C₂₋₆ alkynyl optionally substituted by one or more fluoro atoms or cyano, C₃₋₇ cycloalkyl, or aryl optionally substituted with C₁₋₄alkyl, C₁₋₄alkoxy, halogen, CF₃, nitro, cyano, or CO₂C₁₋₄alkyl; and
R⁵ is hydrogen or C₁₋₄ alkyl.

15. A compound of formula (I) as defined in any one of Examples 1 to 136, or a pharmaceutically acceptable salt thereof.

16. A pharmaceutical composition comprising a compound according to any one of claims 1 to 15, or a pharmaceutically acceptable salt thereof and a pharmaceutically acceptable carrier.

17. A method for the treatment of a disease or condition in which GPR116 plays a role comprising a step of administering to a subject in need thereof an effective amount of a compound according to any one of claims 1 to 15, or a pharmaceutically acceptable salt thereof.

18. A method for the regulation of satiety comprising a step of administering to a subject in need thereof an effective amount of a compound according to any one of claims 1 to 15, or a pharmaceutically acceptable salt thereof.

19. A method for the treatment of obesity comprising a step of administering to a subject in need thereof an effective amount of a compound according to any one of claims 1 to 15, or a pharmaceutically acceptable salt thereof.

20. A method for the treatment of diabetes comprising a step of administering to a subject in need thereof an effective amount of a compound according to any one of claims 1 to 15, or a pharmaceutically acceptable salt thereof.

21. A method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound according to any one of claims 1 to 15, or a pharmaceutically acceptable salt thereof.

22. A compound of formula (XII): 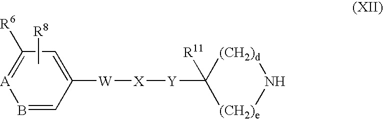
wherein one of A and B is nitrogen and the other is CR¹;
W and Y are independently a bond, an unbranched or a branched C₁₋₃ alkylene or an unbranched or a branched C₂₋₃ alkenylene;
X is selected from CH₂, O, S, CH(OH), CH(halogen), C(O), C(O)O, C(O)S, SC(O), C(O)CH₂S, C(O)CH₂C(OH), C(O)CH₂C(O), OC(O), NR⁵, CH(NR⁵R⁵⁵), C(O)NR², S(O) and S(O)₂;
R¹ is hydrogen, halogen, cyano, C(O)NH₂, C₁₋₄alkyl, SO₂C₁₋₄alkyl, SOC₁₋₄alkyl or SC₁₋₄alkyl;
R² is hydrogen or C₁₋₄ alkyl;
R⁵ and R⁵⁵ are independently hydrogen or C₁₋₄alkyl; or taken together R⁵ and R⁵⁵ may form a 5 or 6 membered heterocyclic ring;
R⁶ is hydrogen, halogen, CN, C₁₋₄alkyl, C₁₋₄alkoxy, ethynyl, C(O)NR⁷R⁷⁷ or C₁₋₄alkyleneS(O)_{f};
R⁷ and R⁷⁷ are independently hydrogen or C₁₋₄alkyl; or taken together R⁷ and R⁷⁷ may form a 5 or 6 membered heterocyclic ring;
R⁸ is hydrogen, halogen, CN, C₁₋₄alkyl or C₁₋₄alkoxy;
R¹¹ is hydrogen or hydroxy;
d is 0, 1, 2 or 3;
e is 1, 2, 3, 4 or 5;
with the proviso that d+e is 2, 3, 4 or 5; and
f is 0, 1 or 2.
